(19) United States
(12) Patent Application Publication
(10) Pub. No.: US 2013/0191033 A1
(43) Pub. Date: Jul. 25, 2013

(54) Analyzing spectra

(71) Applicant: Isis Innovation Ltd., Oxford (GB)

(72) Inventor: Nina Morgner, Oxford (GB)

(73) Assignee: Isis Innovation Ltd., Oxford (GB)

(21) Appl. No.: 13/694,708

(22) Filed: Dec. 26, 2012

Related U.S. Application Data
(60) Provisional application No. 61/631,188, filed on Dec. 27, 2011.

(51) Int. Cl. G01N 33/68; G06F 17/10

(52) U.S. Cl. CPC G01N 33/68; G06F 17/10
USPC 702/19

(57) ABSTRACT

Systems and methods for analyzing spectra are described. In analyzing the spectra, peaks are identified, and complexes and sub-complexes are assigned to their respective peaks.

### CROSS-REFERENCE TO RELATED APPLICATION

This application claims priority to co-pending U.S. provisional application entitled “ANALYZING SPECTRA” having Ser. No. 61/631,188, filed on Dec. 27, 2011, which is entirely incorporated herein by reference.

### CROSS-REFERENCES

Applicant incorporates by reference the following publications as if they were fully set forth herein expressly in their entireties:

“Ultraslow oligomerization equilibria of p53 and its implications,” by Natan, et al., PNAS, vol. 106, no. 34, 14327-14332, 2009 Aug. 25 2009;

“Isoforms of U1-70k Control Subunit Dynamics in the Human Spliceosomal U1 snRNP,” by Hernandez, et al., snRNP PLoS ONE 4(9): e7202doi:10.1371/journal, pone.0007202, published Sep. 28, 2009;

“Mass Spectrometry Reveals Stable Modules in holo and apo RNA Polymerases I and III,” by Lane et al., Structure 19, 90-100, Jan. 12, 2011.

“Mass Spectrometry of Intact V-Type ATPases Reveals Bound Lipids and the Effects of Nucleotide Binding,” by Zhou et al., Science 334(6054):380-385 (2011).

“Heterogeneity and dynamics in the assembly of the Heat Shock Protein 90 chaperone complexes,” Ebong et al., Proc Natl Acad Sci USA 108 (44) 17939-17944 (2011).

“Massign: An assignment strategy for maximising information from the mass spectra of heterogeneous protein assemblies” Morgner, N. and Robinson, C. V., Anal. Chem, 84 (6), 2939-2948, (2012).

Supporting information Massign: An assignment strategy for maximizing information from the mass spectra of heterogeneous protein assemblies, attached hereto as Appendix A.

### BACKGROUND

1. Technical Field

The present disclosure relates generally to spectrometry and, more particularly, to analyzing complex spectra.

2. Description of the Related Art

Conventionally, computer programs have been used to analyze spectra of simple systems. However, conventional programs have limitations, insofar as they are unhelpful in analyzing spectra from large complexes.

### SUMMARY

The present disclosure provides systems and methods for analyzing mass spectra from large complexes. The broadest embodiments include the steps of receiving an experimental mass spectrum from a spectrometer; identifying peak series in the experimental mass spectrum and simulating the experimental mass spectrum by simulating the charge state series of different components determined from identifying peak series; and assigning complexes and sub-complexes associated with the identified peak series.

In another aspect, one or more embodiments include the steps of: identifying peak series and determining mass-to-charge ratios in an experimental mass spectrum; simulating charge state series; and assigning complexes, sub-complexes, and/or kinetics associated with the identified peak series.

In another aspect, one or more embodiments are directed to a system involving use of at least one computing device and at least one application executable in the at least one computing device, the at least one application comprising logic by which the system receives an experimental mass spectrum from a spectrometer; identifies peak series in the experimental mass spectrum and simulates the experimental mass spectrum by simulating the charge state series of different components determined from identifying peak series; and assigns complexes and sub-complexes associated with the identified peak series.

In another aspect, in one or more embodiments of the system, the at least one application comprises logic by which the system: identifies peak series and determines mass-to-charge ratios; simulates charge state series; and assigns complexes, sub-complexes, and/or kinetics associated with the identified peak series.

Other systems, devices, methods, features, and advantages will be or become apparent to one with skill in the art upon examination of the following drawings and detailed description. It is intended that all such additional systems, methods, features and advantages be included within this description, be within the scope of the present disclosure, and be protected by the accompanying claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a flowchart showing steps in a smoothing sub-program.

FIG. 2 is a screen capture of a user interface for the smoothing sub-program of FIG. 1.

FIG. 3 is a screen capture of a user sub-program that allows smoothing of a data set, instead of a single spectrum.

FIG. 4 is a flowchart showing steps in a linearization sub-program.

FIG. 5 is a flowchart showing steps in a background-finding sub-program.

FIG. 6 is a screen capture of the background-finding sub-program.

FIG. 7 diagrams an example of results obtained from the background-finding sub-program of FIG. 4

FIG. 8 is a flowchart showing steps in a sub-program for automatically finding peaks in a mass spectrum.

FIG. 9 is a screen capture of a user interface for the sub-program of FIG. 7, showing a fixed threshold being applied.

FIG. 10 is a screen capture of the user interface for the sub-program of FIG. 7, showing additional thresholds being applied.

FIG. 11 is a flowchart showing steps in a sub-program for finding mass series automatically.

FIG. 12 is a screen capture of the user interface for the sub-program for finding mass series, showing user evaluation of a found mass series.

FIG. 13 is a screen capture of a user interface for the sub-program of FIG. 11.

FIG. 14 is a flow chart showing steps in a sub-program for semi-automatically finding peak series.

FIG. 15 is a flowchart showing steps in a sub-program for fitting mass series to an experimental spectrum.

FIG. 16 is a screen capture showing a user interface for the sub-program for fitting mass series to an experimental spectrum described in FIG. 15.

FIG. 17 is a flowchart showing steps in a sub-program for fitting Gaussians to found peaks and mass series.

FIG. 18 is a screen capture showing a user interface for the sub-program for fitting Gaussians to found peaks and mass series described in FIG. 17.

FIG. 19 is a screen capture showing the user interface of FIG. 18, with a correction for peak overlap function.

FIG. 20 is a flow chart showing steps in a sub-program for adjusting for adducts.

FIG. 21 is a screen capture showing a user interface for the sub-program for adjusting for adducts described in FIG. 20.

FIG. 22 is shows a portion of the user interface of FIG. 21 in greater detail.

FIG. 23 is a flow chart showing steps of a sub-program that can be used to fit a set of spectra which contain the same peak series'.

FIG. 24 is a screen capture showing how to determine a mass shift for using the sub-program described in FIG. 23.

FIG. 25 is a screen capture showing a sub-program in which the user can input a fit parameter to fit a set of spectra as described in FIG. 23.

FIG. 26 is a flow chart showing steps in a sub-program for fitting mass series, a simulated spectrum, an experimental spectrum and identifying and accounting for missed peaks.

FIG. 27 is a screen capture of a user interface for the sub-program of FIG. 26.

FIG. 28 is a screen capture showing the use of the Fit Gaussian sub-program described in FIG. 17 and FIG. 26 to identify and account for missed peaks.

FIG. 29 is a flow chart showing steps and sub-programs used to assign complexes and/or sub-complexes to a found mass series.

FIG. 30 shows a screen capture of a user interface for a sub-program to set up component spectra, that can be used to assign complexes and/or sub-complexes to a found mass series.

FIG. 31 shows a portion of the user interface of FIG. 30 in greater detail.

FIG. 32 shows a screen capture of a user interface for a sub-program to find possible subunit combinations, by which two complexes differ.

FIG. 33 shows a screen capture of a user interface for a sub-program to find possible subunit combinations that can be used to assign complexes and/or sub-complexes to a found mass series.

FIG. 34 shows a screen capture of a user interface that can be used to calculate the mass of a theoretical complex

FIG. 35 shows a portion of the main call screen of FIG. 27, showing a user interface to access the sub-program to find possible sub-unit combinations and a user interface to access a sub-program to calculate the theoretical mass of a found peak mass.

FIG. 36 shows a flow chart showing the steps in a sub-program for following complex kinetics.

FIG. 37 shows a screen capture showing a user interface for the sub-program for following complex kinetics.

FIG. 38 shows a flow-chart showing the steps in smoothing a step-function.

FIG. 39 shows a screen shot of a sub-program that allows looking through a number of spectra either all in one folder or in a set of folders.

FIGS. 40A and B depict an exemplary embodiment of the present disclosure in which: (A) we first identify all mass series in the spectrum (blue lines) and (B) assign complexes to masses and develop dissociation network (green lines).

FIG. 40C depicts a list of sub-programs that may be included in an exemplary software package or logic of the present disclosure and an order in which they may be implemented.

FIGS. 41A and B depict for an assignment example: (A) components of mass spectra that were simulated, and masses and charge states that were determined, and complexes not yet identified at the given stage depicted; and (B) a schematic used to derive values for component mass spectra.

FIG. 42 depicts in the main graph the mass shift of a complex stemming from adducts attached to the surface of the complex, which correlates the mass shift with the mass, and in the inset the surface area per mass calculated for exemplary globular proteins for the assignment example of FIGS. 41A and B.

FIGS. 43 A-E depict an assignment process using an embodiment of the present disclosure after relationships between complexes are established from FIGS. 41 A and B in which the relationships can be used in the final assignment of the complexes. FIG. 43 (A) depicts an assignment process for an exemplary complex 5, a solution complex of mass 387 356 Da. In the example, the assignment process reduces the possibilities from 385 (FIG. 43 B) or 474 (FIG. 43C) to two. Assignment of complex 4 (FIG. 43D) reduces the 538 possibilities to one, which decides the final assignment to the series shown in FIG. 43E.

### DETAILED DESCRIPTION OF THE EMBODIMENTS

Reference is now made in detail to the description of the embodiments as illustrated in the drawings. While several embodiments are described in connection with these drawings, there is no intent to limit the disclosure to the embodiment or embodiments disclosed herein. On the contrary, the intent is to cover all alternatives, modifications, and equivalents.

Conventionally, computer programs have been able to analyze spectra of simple systems, but have been largely unhelpful in analyzing spectra from large complexes. For example, commercial mass spectrometry (MS) software was developed to investigate small proteins or peptides. It can identify charge state series of small protein complexes, providing the charge state series are sufficiently separated. This will likely be the case for relatively small complexes containing only a few subunits. It may be possible to dissociate and therewith reveal the identity of one or two sub-units. For larger complexes, the knowledge of one or two subunits is not sufficient. The challenge of assigning complexes and their sub-complexes increases with the number of subunits and therewith potential subunit combinations.

For mass spectra of protein mixtures a possible assignment approach is spectral deconvolution. For mass spectra that are complex and/or contain multiple different species with many overlapping charge states, this approach becomes problematic especially for large protein complexes, for which wide mass ranges have to be covered and peaks are often broadened due to incomplete desolvation.

For large heterogeneous systems such as the rotary ATPase exemplified below, the identity of the complexes in solution was unknown at the time of our study. One objective, among others, of the present systems and methods is therefore their complete assignment. The approaches mentioned above are not applicable in these cases.

The present disclosure, therefore, provides systems and methods for analyzing spectra from large complexes. The present disclosure includes an assignment strategy that provides for the analysis of complicated spectra from heterogeneous, high mass complexes among other complexes. This strategy involves steps of assignment comprising: identification of charge state series and, hence, determination of their masses and their subsequent assignment to complexes.

Broadly, the disclosed embodiments teach the steps: of identifying peak series and simulating charge state series in an experimental mass spectrum; and assigning complexes, subunit combinations, and/or kinetics associated with the identified peak series. For example, the step of identifying peak series can be done including simulation of the component mass spectra for all complexes present in a spectrum, so that the sum of these component spectra resembles most closely the experimental spectrum. The complexes can include for example, proteins. The output from this first step can then be used together with knowledge of the subunit composition/connectivity of the complex determined from the other steps to determine the identity of the (sub)-complexes appearing in a mass spectrum.

In an aspect, one or more embodiments provide a system involving use of at least one computing device and at least one application executable on the at least one computing device, the at least one application comprising logic by which the system: receives an experimental mass spectrum; identifies peak series and simulates charge state series in the experimental mass spectrum; and assigns complexes, subunit combinations, and/or kinetics associated with the identified peak series. For example, the step of identifying peak series can be done including simulation of the component mass spectra for all complexes present in a spectrum, so that the sum of these component spectra resembles most closely the experimental spectrum. The complexes can include for example, proteins. The output from this first step can then be used in the system together with knowledge of the subunit composition/connectivity of the complex determined from the other steps to determine the identity of the (sub)-complexes appearing in a mass spectrum.

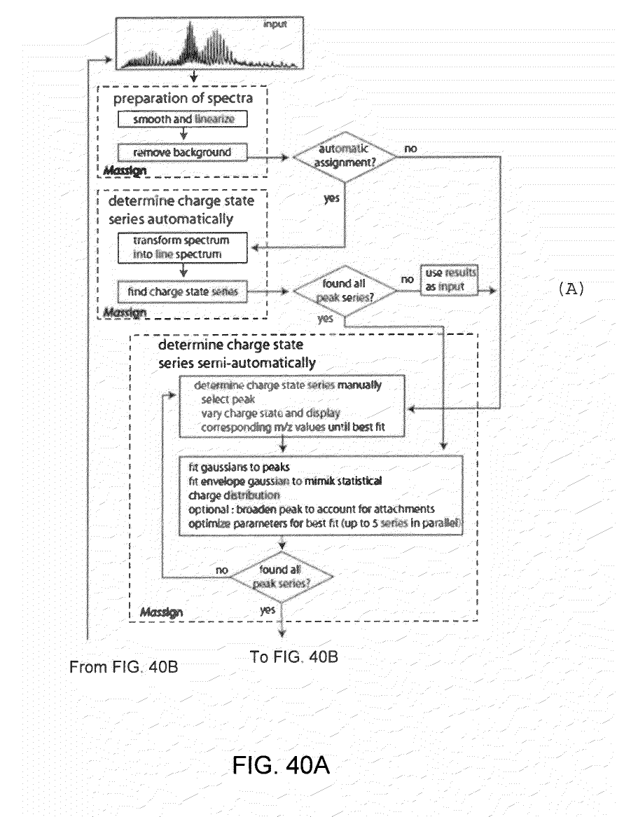

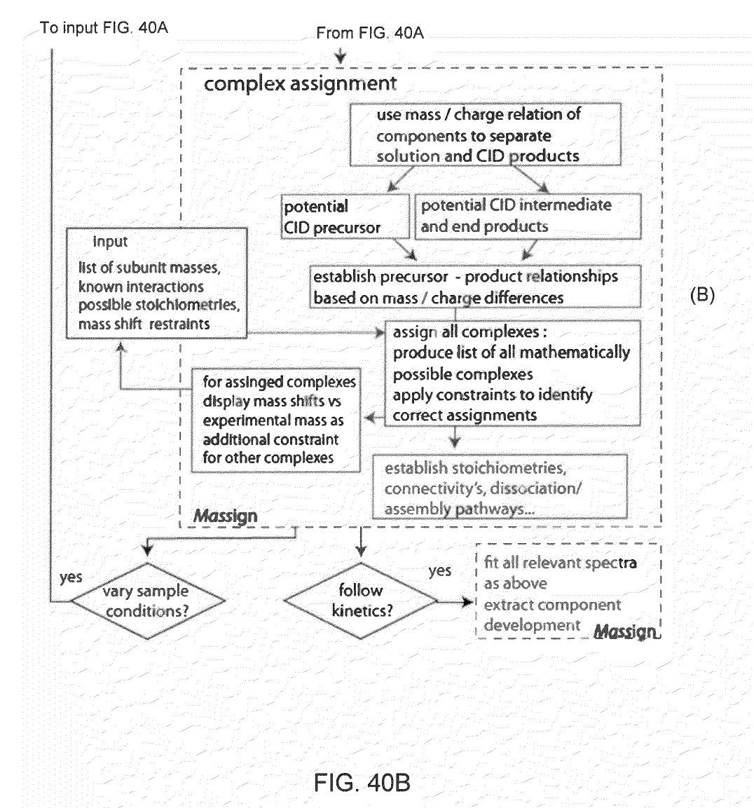

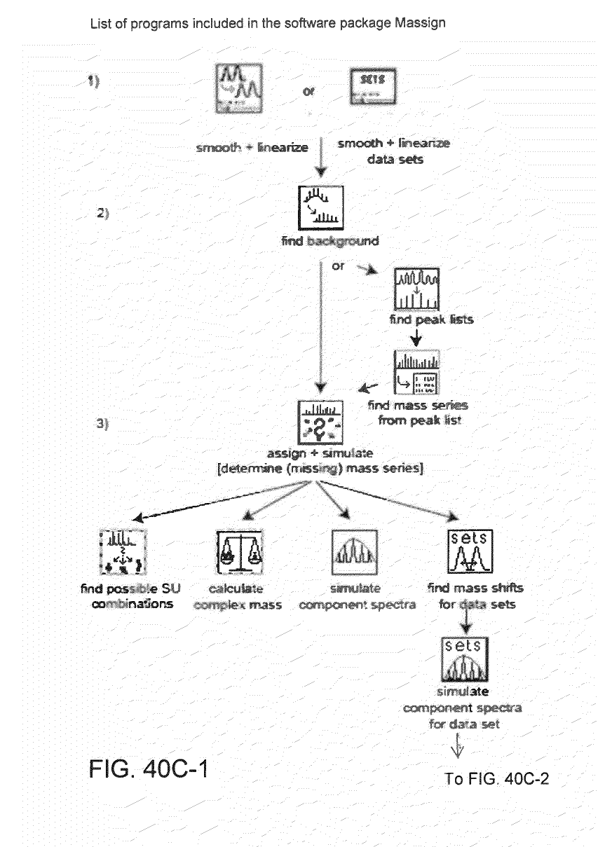

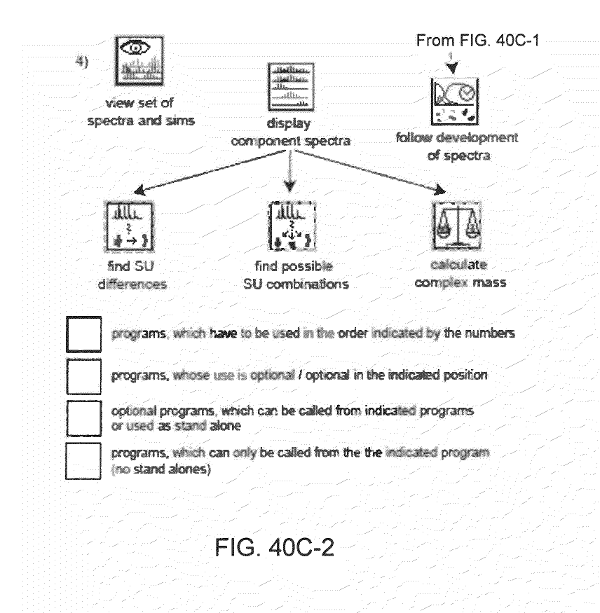

FIG. 40 depicts an exemplary embodiment of the in which: (A) we first identify all mass series in the spectrum (blue lines) and (B) assign complexes to masses and develop dissociation network (green lines).

A) Identifying Peak Series

The present disclosure offers an automatic as well as a semiautomatic approach to identifying peak series and charge state series present in a complex. As an example, protein complexes can carry with them in the gas phase many buffer molecules giving rise to rather broad peaks, with the mass of the naked protein being represented rather by the onset of the peaks, while the peak tops correspond to the complex with adducts attached. In an embodiment to address this situation we can aim at the masses determined from the peak tops the additional mass of the adducts can be taken into account at a later state, during assignment. For both automatic and semiautomatic routines, the approach can be similar in that one peak of the series can be chosen (automatic: likely the most abundant). The charge state of this peak can be varied and the theoretical charge state distribution compared with all the peaks in the spectrum. While the automatic routine can transform the experimental spectrum into a line spectrum, prior to assignment, and select for every peak charge state charge series which fits best. The semiautomatic routine can allow the user to evaluate the best fit by comparing theoretical peak positions with the experimental spectrum. Since the deviation between theoretical peak positions of different possible charge state distributions increases at either end of the charge state distribution, the correct assignment can readily be identified in comparison with the experimental peak positions, even for broad peaks.

B) Simulating Charge State Series

The charge state series of the different components determined can then be simulated. The simulation of each component in the mass spectrum can be a series of peaks whose intensities follow a Gaussian distribution, to mimic the statistical distribution of the charges. The peak shape used for simulation of the individual peaks can also be Gaussian unless the peaks are distorted by small molecule binding (see below). Overlapping charge state series can be considered simultaneously for the simulation process to avoid over-representation of the ion signal where peaks overlap. These simulated spectra can then be displayed simultaneously/overlaid with the experimental spectrum for further inspection. This approach has the advantage that peaks, which were completely or partially overlapping an/or low abundant can become apparent.

Simulation of the component spectra allows use of the whole range of charge states present in the spectrum to determine the correct charge distribution and mass. Inclusion of more charge states increases confidence that the correct charge state and hence mass has been determined. A second advantage is that more realistic mass errors are derived. In an embodiment an approach taken is to determine the correct charge series and then fit each peak to a Gaussian, which determines the midpoint of each peak in the entire charge state series. The standard deviation of the masses of the complex derived from each charge state can then be used as a mass error.

FIG. 40A shows steps that can be involved for simulating a spectrum. In an exemplary embodiment the steps can include:

(1) Smooth and linearize. Spectra can be smoothed to reduce noise and transformed to a linear x-axis.

(2) Combine spectra. Spectra can optionally be combined to reduce noise.

(3) Subtract background.

(4) Find or analyze mass series. This can be done in an automated or semiautomatic way, depending on the quality of the spectra.

(5) Simulate component spectra. Component spectra can be simulated individually. The parameters can be optimized to minimize deviation of the sum of the simulations from the experimental data. In an embodiment this can be done for up to five components in parallel. Further components can be fit in a second fit round.

(6) Obtained spectra can be overlaid with the experimental spectrum, to make visible which parts on the spectrum are not yet accounted for.

(7) Steps 4-6 can be until all components are simulated.

The output from this first part can be a list of masses/charge distributions found in the spectrum, the component spectra and the overall simulation. These can be used as input for the next part, where the aim is to assign (sub) complexes to the components identified by their masses.

Simulating the spectra in the manner described above will be sufficient for many cases, where spectra are well-resolved and qualitative rather than quantitative analysis is required. It may not always be possible to obtain well-resolved spectra. Peak broadening is commonly experienced as a result of water/buffer molecules, which stay attached to the complexes, particularly when efforts to desolvate them result in the dissociation of the complex.

A problem can then be the asymmetry of broadened peaks. The trailing edge of one peak can mask an additional peak, or add to the intensity of the second peak. In an embodiment, we can add adducts to the peak simulation. One way of doing this is by replacing the trailing edge of every simulated peak by a broadened version of the same peak. The optimization parameter is termed the “broadening factor”. The present disclosure can determine the broadening factor, which optimizes the agreement of the experimental spectrum and the simulation via a minimization of the root-mean-square deviation (rmsd). If the user recognizes the need, the broadening factors for the different components can be varied independently. This may not be necessary, even if intuitively one might expect differences in desolvation of solution complexes and those formed via collision induced dissociation (CID). Nevertheless complexes observed within one spectrum under the same experimental conditions will have experienced the same desolvating conditions, independent, if these led to CID or not.

C) Assigning Complexes

Once the masses and charges of the components in a mass spectrum are determined, we then assign these to the correct complexes. Knowing the mass of a complex will provide sufficient information to distinguish between a monomer and a dimer of a known protein or to establish whether or not a ligand is bound to a complex. Determining the composition of a complex with a range of subunits of unknown stoichiometry is much more challenging. In an embodiment, we determine a list of mathematically possible complexes, based on the masses of the subunits (preferably masses determined by LC/MS or seen in isolation in an ESI spectrum). The list may include all mathematically possible complexes. If only genome sequence data is available and post-translational modifications are unknown, the user may want to keep in mind a possible systematic mass error in the assignment process. The list of potential assignments, which can have several hundred entries, can then be reduced by ruling out those which are known to be biologically impossible, due to compositional data from proteomics, cross-linking experiments, tandem-MS, etc. A list of rules can be compiled such that complexes that do not fulfill the known requirements are excluded.

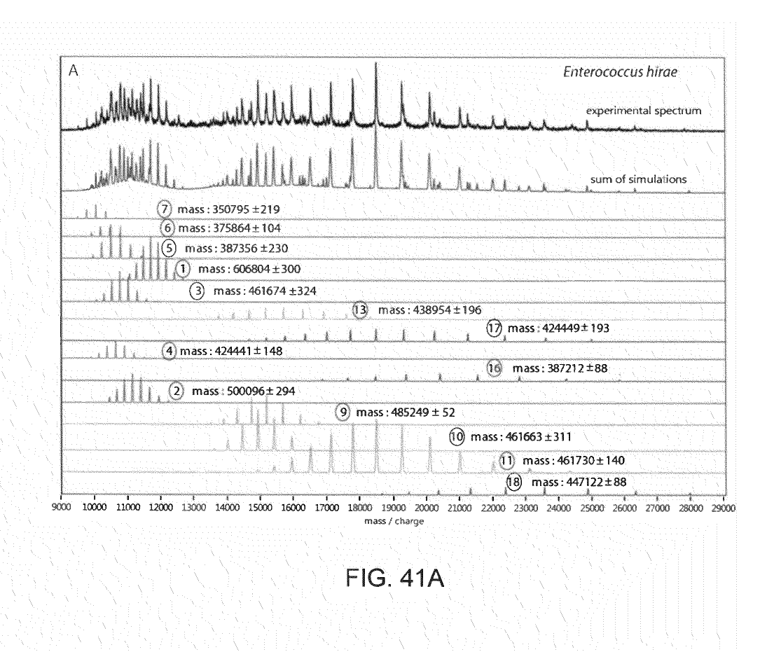

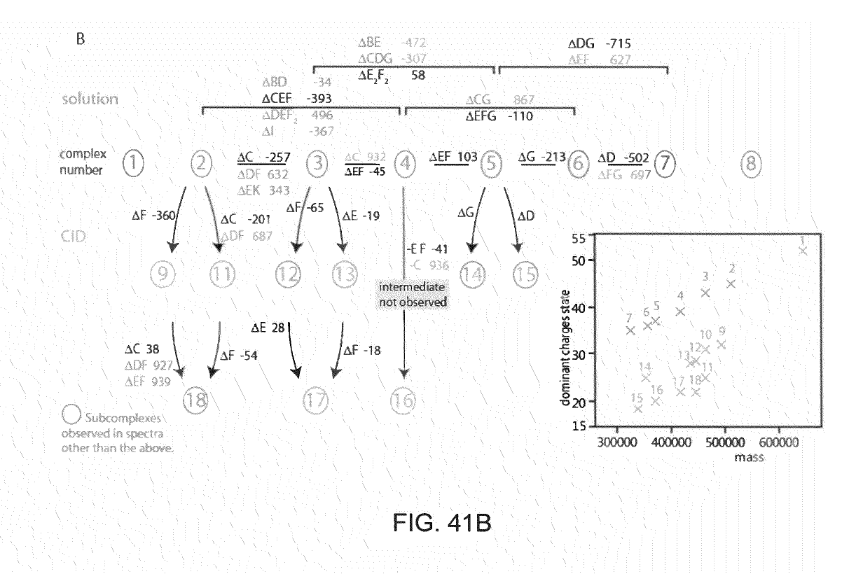

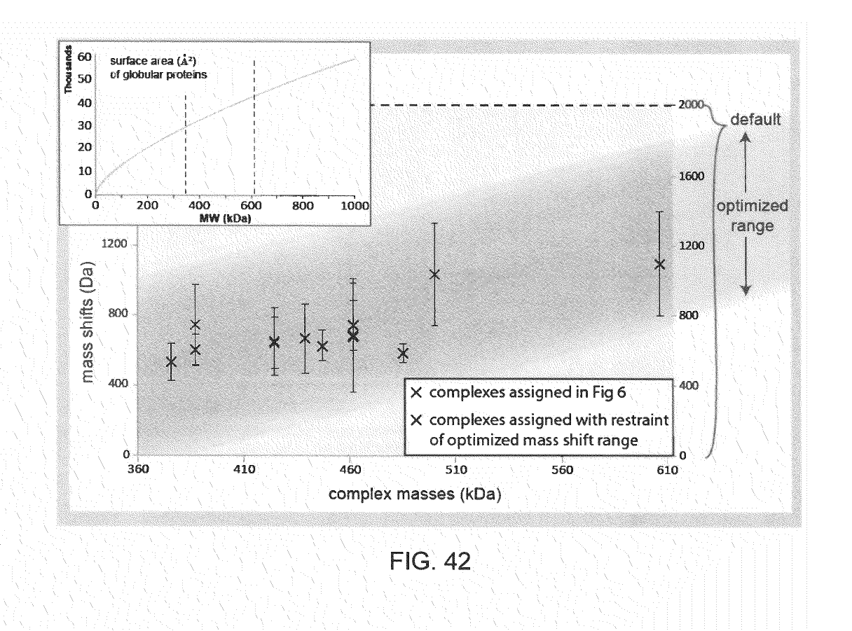

A feature of the present disclosure may include distinction between complexes formed in solution and those formed via CID. This can be achieved on the basis of their mass to charge correlation. Complexes that result from CID will have lost a higher proportion of the overall charge and appear at lower charge values on a mass/charge plot than the same complexes formed in solution (see inset in FIG. 41B). If complexes lose a subunit via CID, in general this process does not go to completion and as such 100% of the complex will not dissociate. Some of the original complex will remain. As a consequence the complex will be present as both the precursor and product complex. Complexes therefore have CID relation-ships which can be established, even if the identity of the complexes is as yet unknown.

In a comparable fashion, different solution complexes emerge from each other by losing subunits or sub-complexes. These relationships can be established likewise. Differences between complexes can be used as restraints for the assignment (e.g., a subunit must/must not be present in the precursor/product complex). In many cases, some restraints can be based on previous research. For example, a sub-complex of the intacting complex may have been crystallized or cross-linking experiments may have revealed neighboring relationships between two proteins. These rules, as well as the maximum copy number of each protein subunit (if known), can be used as input into the assignment module.

The increase of the measured mass compared to the mass of the naked complex is another parameter that can be considered during mass determination. The measured mass increases proportionally with the size of the complex, due to attachment of buffer and water molecules. For example, for a complex of several hundred kilodalton, this mass shift can easily be ˜2000 Da. This number should not be treated as an error since such a sizable error would lead to too great an ambiguity in assignment. This mass shift generally follows certain rules. The extent of attachment depends on the surface area of a complex, which in turn correlates to the mass of the complex. All complexes within one spectrum will experience the same conditions in solution (buffer conditions) and in the gas phase (desolvation process). Their mass shifts therefore scale linearly with the surface area of the protein complexes to which adducts can attach. The overall shape of large complexes is to a rough approximation globular which correlates the mass shift therewith with the mass of the complex (see inset in FIG. 42). This correlation can still be of use for real complexes, which are usually not globular. Assignment of one or two complexes in a spectrum therefore can define the mass shifts to be expected during the assignment of further complexes. In general sub-complexes of very high as well as of low mass will be the easiest to assign. A sub-complex of approximately half of the mass of the intact complex will have a much larger list of potential subunit combinations compared to a complex, which has lost only one or two subunits. Consequently, if assignment of the complexes proves difficult with the default mass shift of 2 kDa, these “easy to assign” complexes are a logical choice as starting point in the assignment process and then define the mass shifts to be taken into account for other complexes in the mass spectrum.

Reducing the potential subunit combinations for each complex will often leave very few possibilities. The next step can be to evaluate the likelihood of each of these possibilities to be the correct one. The sub-complexes forming from one complex do not represent a collection of random complexes but, rather, will be related to each other according to gas phase as well as solution dissociation patterns. So can a stable sub-complex that was found to dissociate in a pairwise manner in solution in one case be expected to show this behavior for all applicable complexes. (Example: if we observe solution complexes A₂B₂CDE and ABCDE, we can conclude AB is readily lost and lost in pairwise interaction. If we then as well observe A₂B₂CD, we would expect the same rule to be applicable and see ABCD). Equally a subunit that readily dissociates under CID in one complex can be expected to dissociate from all solution phase complexes containing this subunit. If we observe the solution complex ABCD and CID complex ABC (loss of D), the observation of solution complex BCD would suggest the existence of the BC complex, formed by CID.

These patterns can be defined during the assignment process and give insights into the behavior of the complexes as well as aiding the assignment process, by establishing a self-consistent set of complexes, which give rise to the observed spectrum. This is explained in more detail below using the assignment of the rotary ATPase from E. hirae as a worked example.

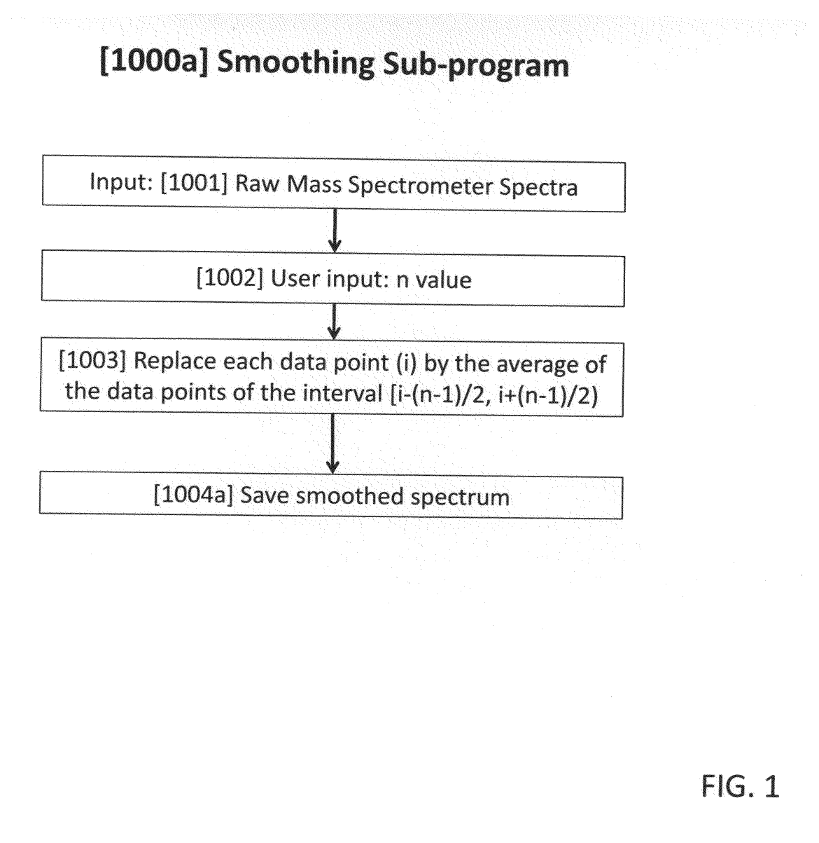

Details of various systems and methods for analyzing complex spectra are now described with reference to FIGS. 1 through 39, below.

In an embodiment, the present system can include a computer program that can be a module based program comprising multiple sub-programs in which data can be accessed and analyzed. The particular order of sub-program use is dependent on the needs of a user with respect to a particular data set. While the embodiments described below describe the modular components being accessed in a particular order, there is no intent to limit the disclosure to the access order disclosed herein. On the contrary, the intent is to cover all alternative orders of sub-program access and use. Furthermore, use of any particular sub-program is optional and is at the discretion of the user.

FIG. 1 is an exemplary flow chart showing steps in a smoothing sub-program 1000a. Typically, a mass spectra signal contains noise, which disrupts analysis. To filter out this noise, 1001 raw mass spectrometer spectra can be smoothed using 1000a a smoothing sub-program. As shown in FIG. 1, in one embodiment, the smoothing sub-program receives 1002 a smoothing constant, n, from the user, and each data point, i, from the data that has been input 1001 from the mass spectrometer, is replaced 1003 by an average of the data points in the interval:

[i−(n−1)/2, i+(n−1)/2)].

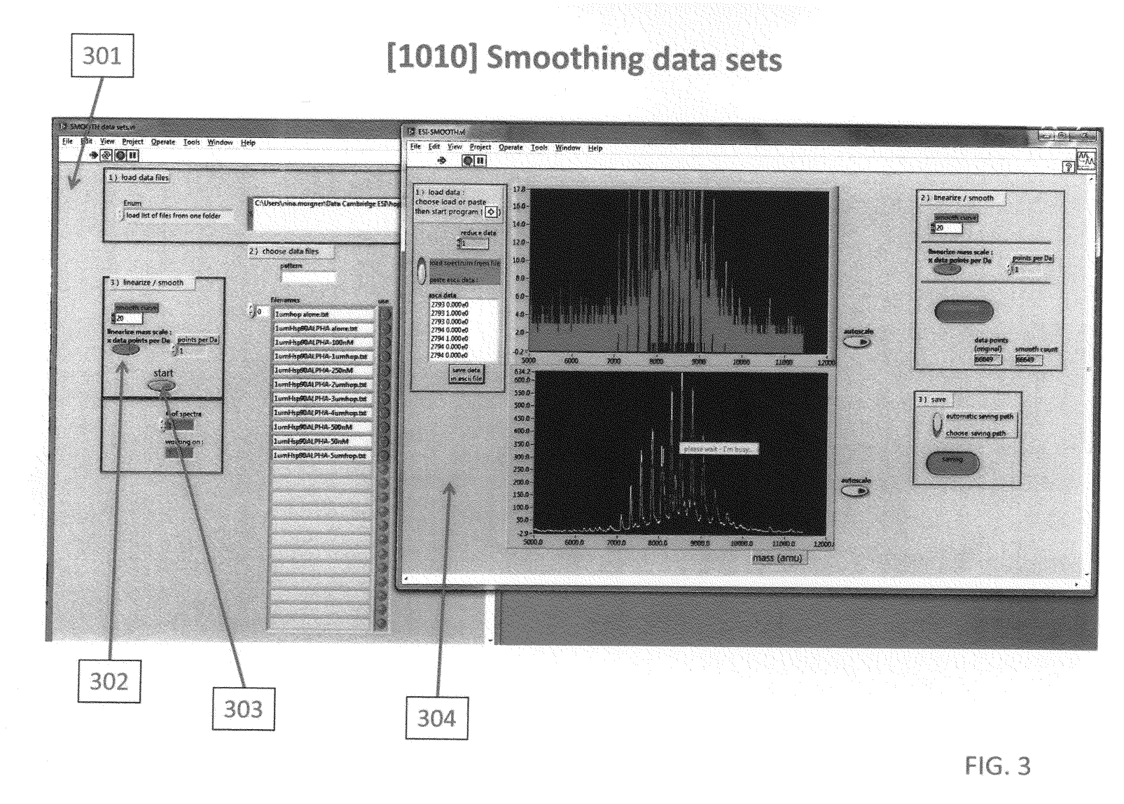

This results in a smoothed spectrum that is subsequently saved 1004a by the smoothing sub-program 1000a. That smoothed spectrum can be used in a linearization sub-program 2000 of FIG. 3.

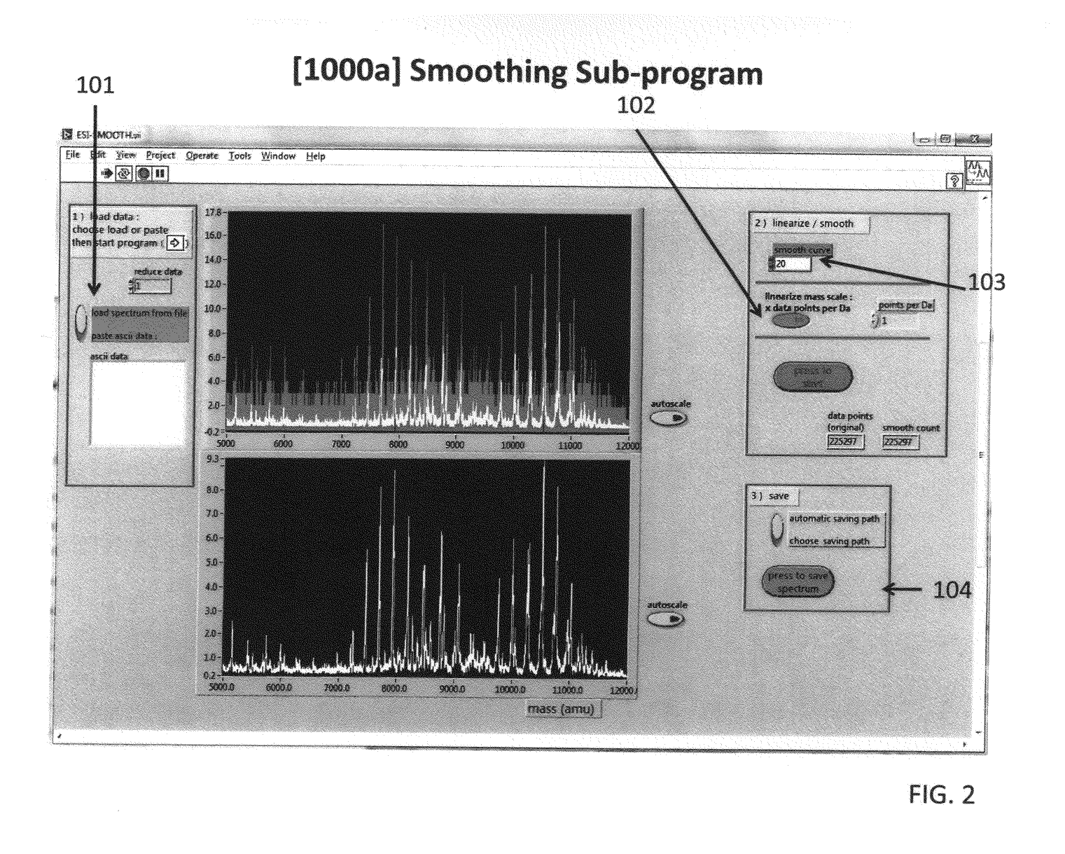

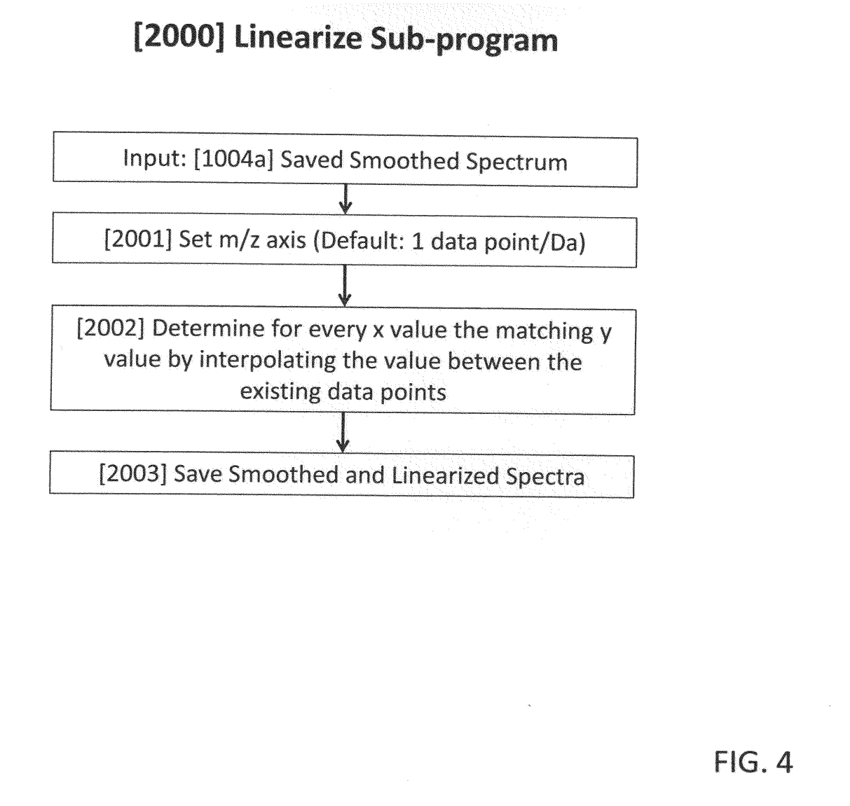

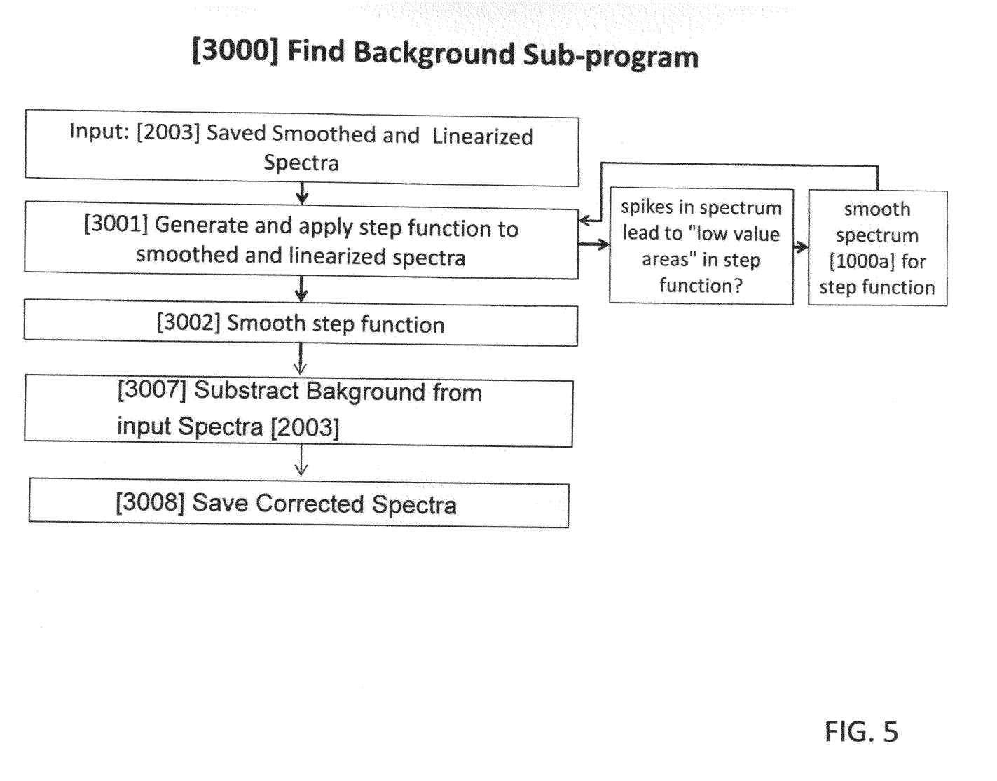

FIG. 2 is a screen capture of a user interface for the smoothing sub-program. There can be an interface 101 that allows the user to load data into the sub-program. There can also be an interface 102 that allows the user to utilize a linearization sub-program 2000 (FIG. 4), a smoothing sub-program 103 [1000a] and a find background sub-program 3000 (FIG. 5). There is also an interface 104 that allows the user to save spectra.

FIG. 3 is a screen capture showing a program that allows loading of a set of spectra 301, which then can be smoothed and linearized with the same parameters 302. When the user presses “start” 303 the program opens the smoothing program 1000a (304) as a sub-program and smoothes/linearizes and then saves all spectra.

FIG. 4 is a flow chart showing steps in an exemplary embodiment of the linearization sub-program 2000. As described above, the linearization sub-program 2000 can receive as its input the smoothed spectrum from FIG. 1. The linearization sub-program 2000 sets 2001 the mass-to-charge (m/z) axis. While a default value of one (1) data point per Dalton is used, that default value can be changed by the user as needed. Upon setting 2001 the m/z axis, the linearize sub-program can determine 2002 a matching y-value for every x-value. This can be done by interpolating the value between existing data points. The resulting linearized spectrum can then saved 2003.

FIG. 5 is a flow chart showing steps in a 3000 background-finding sub-program. Smoothed and linearized spectra can be loaded into the 3000 background-finding sub-program. The background can be estimated by 3001 generating a step function with a step size (m), chosen by the user, lying under the spectrum. One embodiment of a generated step function 3001 is shown in FIG. 7. To generate the step function 3001, every data point, i, is replaced by the smallest value present in the interval of m data points such that:

i:=min([i−(m−1)/2, i+(m−1)/2]).

Optionally, the step size m can be scaled by increasing the m/z value (k) by choosing a scaling value(s) that renders the step size mₖ₋₁, so that mₖ₋₁=mₖ+i*s.

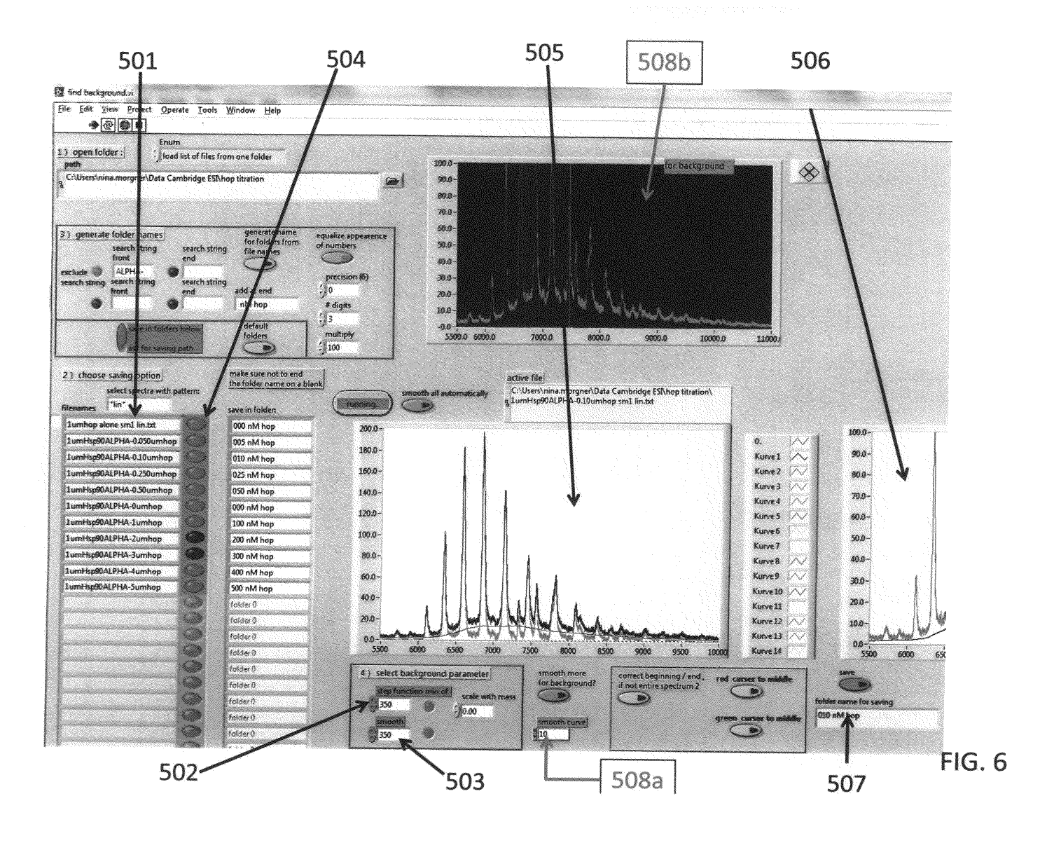

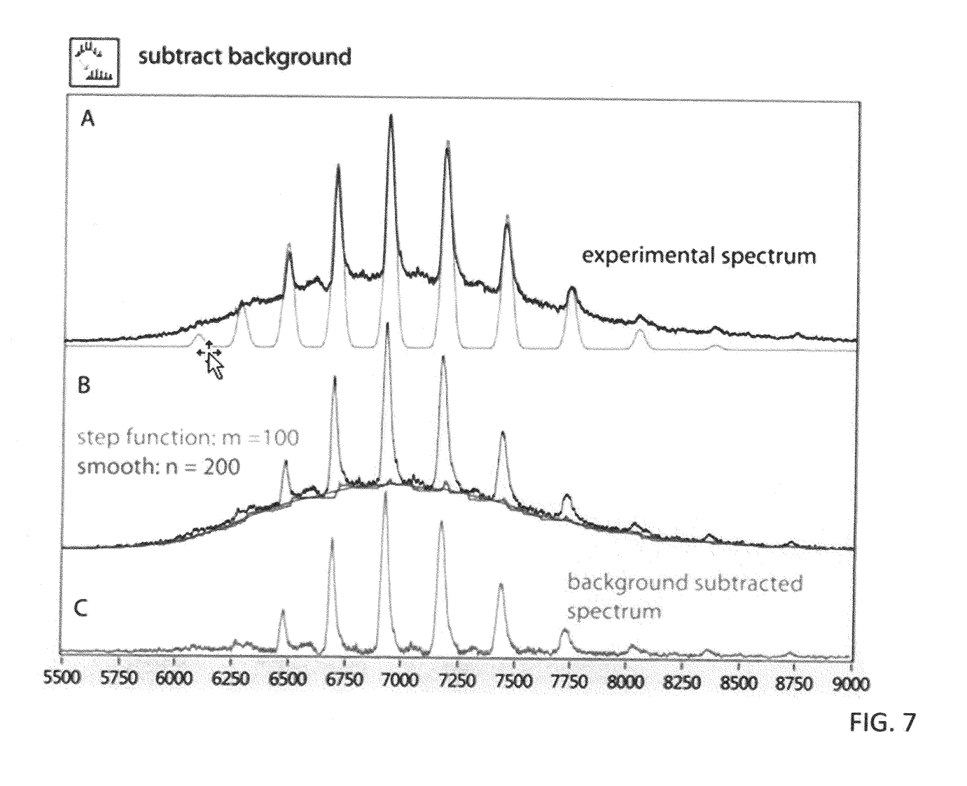

After the step function is generated 3001, the step function can be smoothed 3002 by utilizing a smoothing sub-program 1000b. This smoothing sub-program 1000b can be accessed from the finding background sub-program 3000. This access point is shown in greater detail with reference to FIG. 6. In one embodiment the step function is smoothed by replacing every data point, j, by the average of data points in the interval of n data points, such that:

j:=[j−(n−1)/2, j+(n−1)/2]

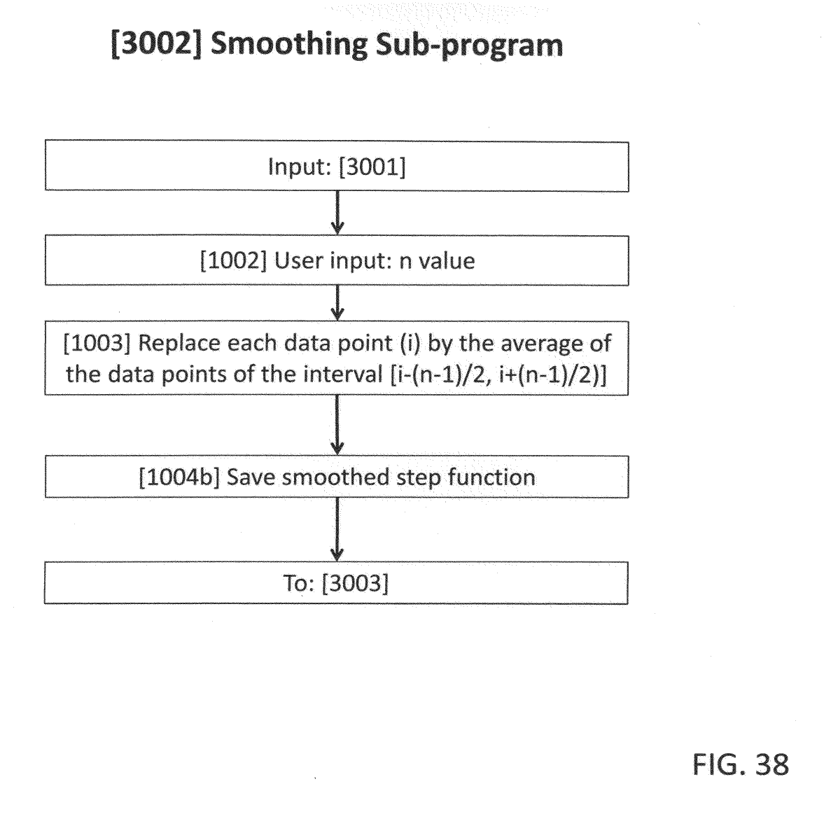

The steps involved in smoothing the step function 3002 are shown in greater detail with reference to FIG. 38.

If the spectrum is not highly smoothed, noise can be the cause of signal spikes pointing down, which can cause the step function in [0053] to be at low value for m points. If this is the case the user has the option to smooth 1000a the spectrum (again) for use of background finding. This only affects the background. The spectrum itself remains the same.

The background can then be subtracted [3007] from the input spectra [2003]. The smoothed and linearized spectra corrected for background are saved 3008. A screen capture showing a user interface for the background-finding sub-program is shown in FIG. 6. An examplary step function and smoothed step function are described with reference to FIG. 5 is shown in FIG. 7.

FIG. 6 is a screen capture of a user interface for the background-finding sub-program. Saved spectra can be loaded into the program and file names of loaded spectra can be displayed 501. The user can choose which spectra to subtract background from by selecting 504 individual loaded spectra from the list of loaded spectra 501. The step function can be generated 502 and smoothed 503 from this interface. If necessary the user has the option 508a to additionally smooth the spectrum 508b to be used to calculate the background step function. The step function and smoothed step function can be displayed 505 and 506. Corrected spectra can be saved 507 from this interface.

FIG. 7 diagrams an example of results obtained from the background-finding sub-program described in greater detail in FIG. 4.

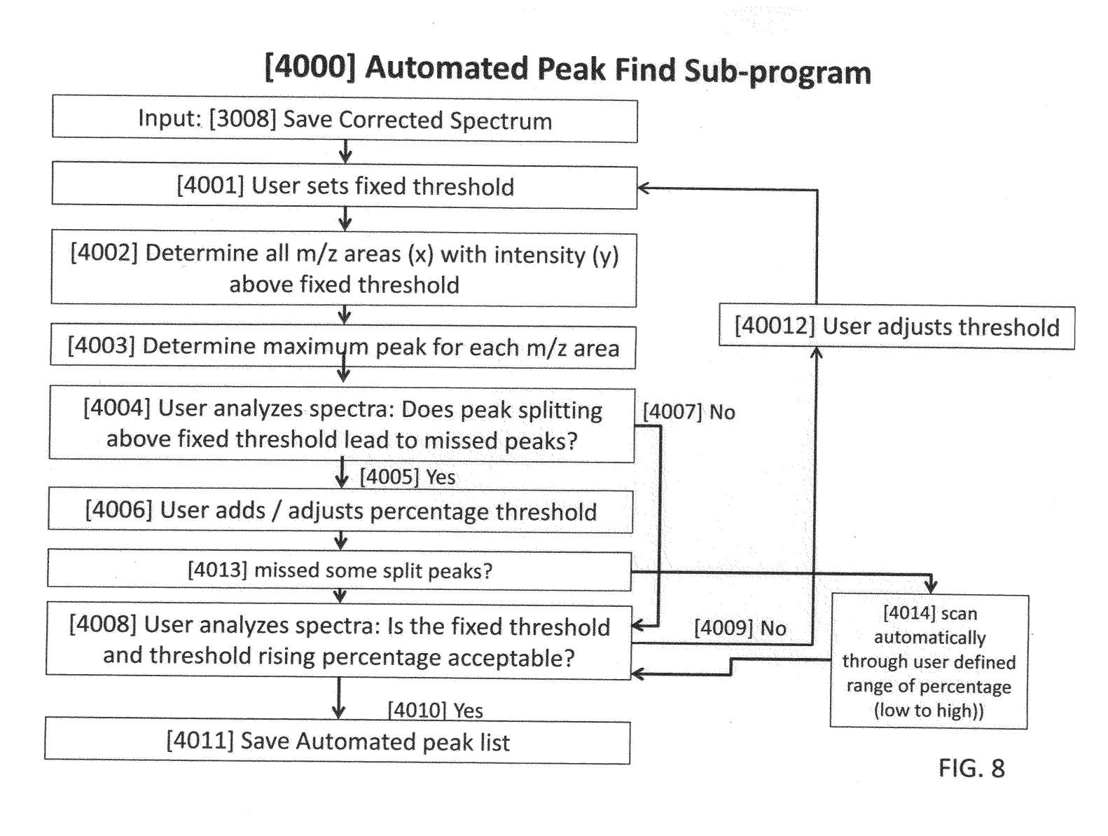

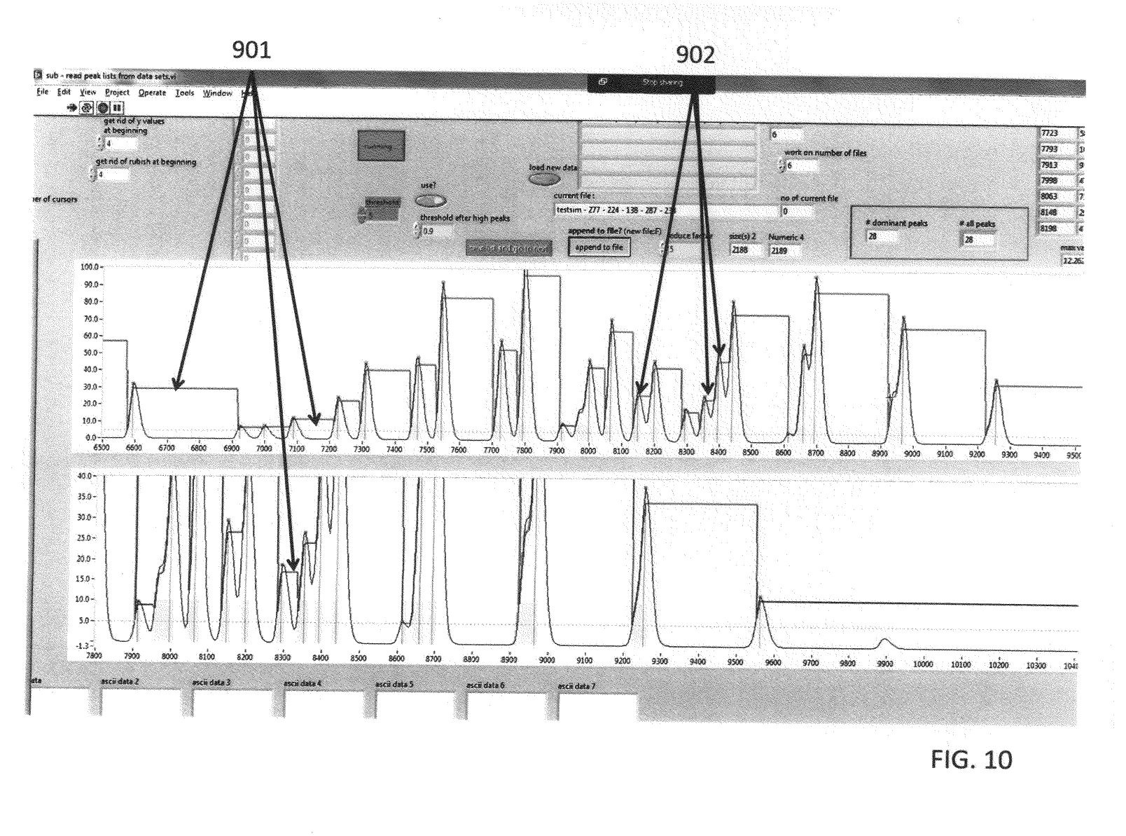

FIG. 8 is a flow chart showing steps in one embodiment of a sub-program 4000 for automatically finding peaks in a mass spectrum. If the spectrum is well resolved this sub-program works to automatically find peaks in the smoothed, linearized and background corrected spectra 3008. To find peaks the user can set 4001 a fixed threshold value. The default is five percent of the highest peak but the user may adjust this as necessary. The sub-program 4000 determines 4002 all m/z areas (x) with intensity (y) that are above the fixed threshold and then determines 4003 the maximum peak for each m/z area. The user can then analyze 4004 the spectra and determine if there is any peak splitting above the fixed threshold that has led the sub-program 4000 to miss peaks. If the user determines 4005 that the fixed threshold has led to peaks being missed, the user 4006 can activate another threshold, a percentage threshold, which raises the threshold after every already determined peak to a certain percentage of the already determined peak. The additional threshold default value can be set at, for example, ninety percent of the height of the peak immediately prior to the m/z area being investigated by the sub-program 4000 for additional peaks. The fixed threshold and percentage thresholds are also shown in FIG. 10.

If a single percentage threshold still misses peaks, the user can activate a threshold scan [4014]. The program can increase the percentage threshold between user defined values and with user defined step size and remember all additional peaks/m/z area for each peak. The sub-program can then determine 4003 the maximum peak for each m/z area according to the adjusted threshold. The user can then analyze 4008 the spectra again to determine if the fixed threshold and the percentage threshold are acceptable or need to be varied. If the user analyzes the spectra 4004 and determines 4007 that no peaks are missing then the user can analyze 4008 the spectra to determine if the thresholds are acceptable.

If the user determines that the fixed threshold and percentage threshold are not acceptable 4009, the user can then 4012 adjust the thresholds. If the user finds the fixed threshold and percentage threshold acceptable, then 4011 the automated peak list can be saved.

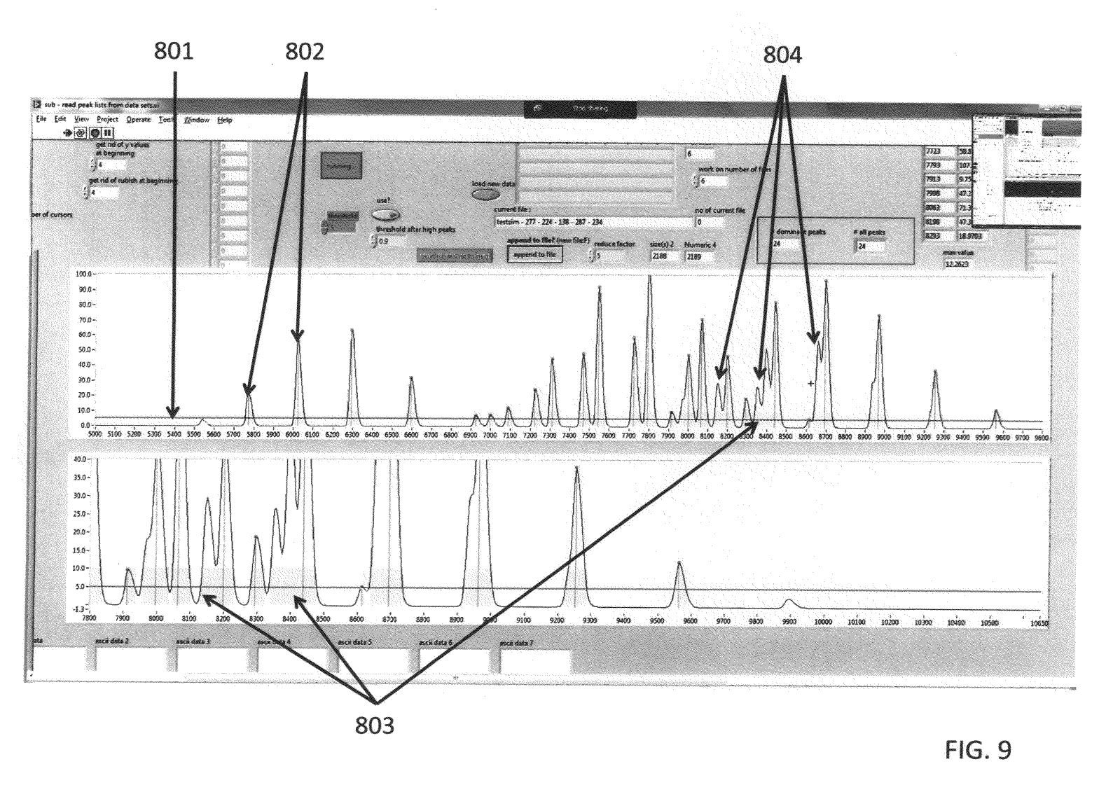

FIG. 9 is a screen capture of a user interface for the automated peak find sub-program with only a fixed threshold being applied. The peak find sub-program finds peaks 802 in a m/z area 803 determined 4002 by the sub-program 4000 that are above a fixed threshold 801. The user can analyze 4004 this spectra and identify missed peaks 804.

FIG. 10 is a screen capture of a user interface for the automated peak find sub-program with additional percentage threshold being applied. This example is the same data set presented in FIG. 9. In this figure the user has adjusted the percentage threshold 4006 to account for missed peaks 804 (FIG. 9). The horizontal dotted line is the fixed threshold chosen by the user 4001. The additional adjusted thresholds 901 are applied to identify previously missed peaks. Peaks missed 804 (FIG. 9) are now identified 902 by the sub-program 4000.

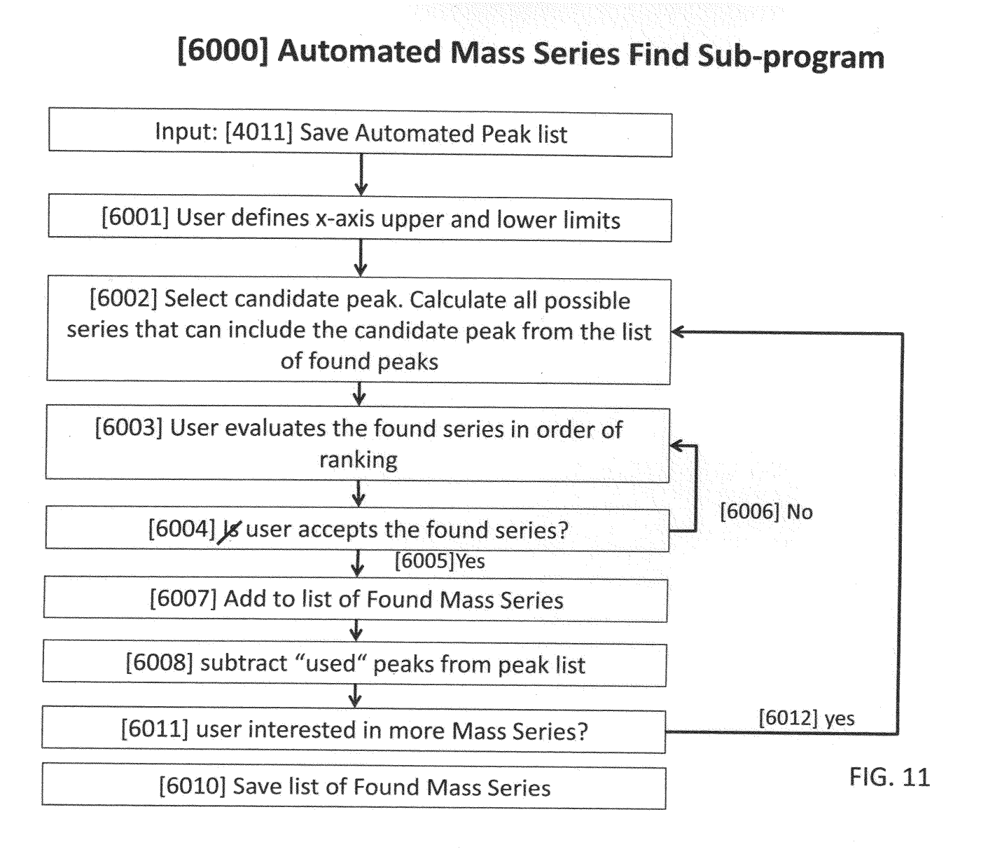

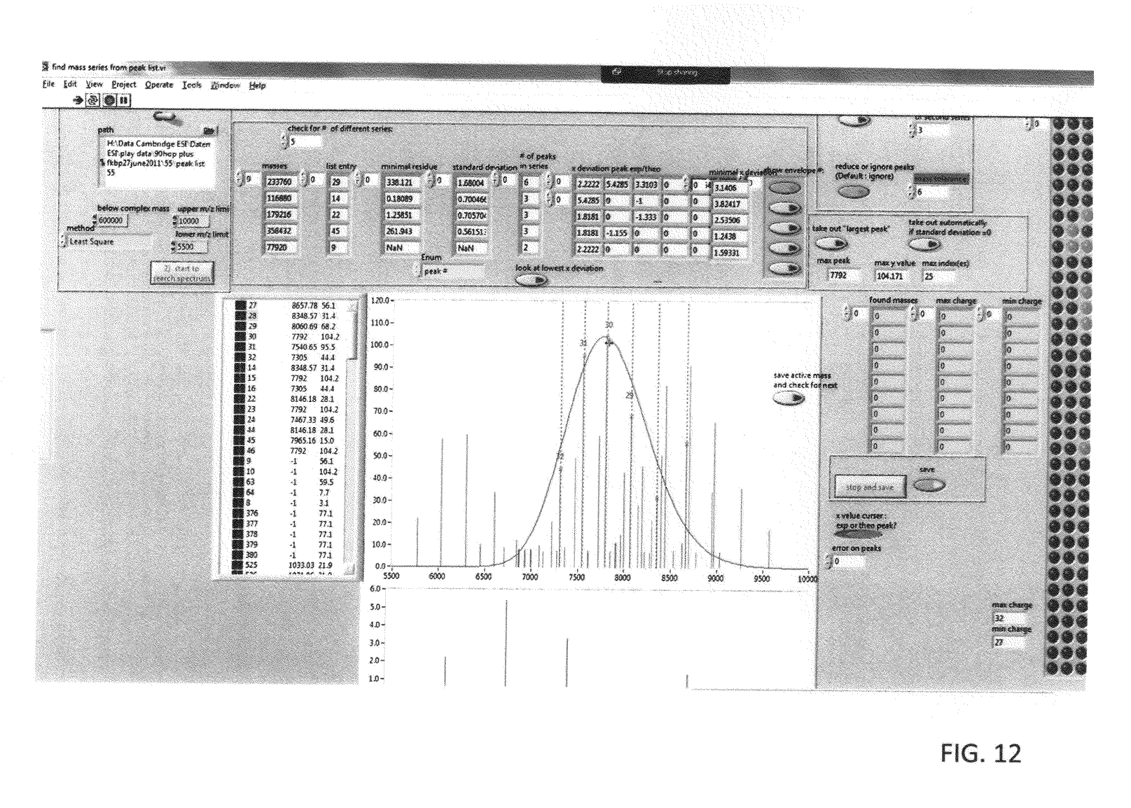

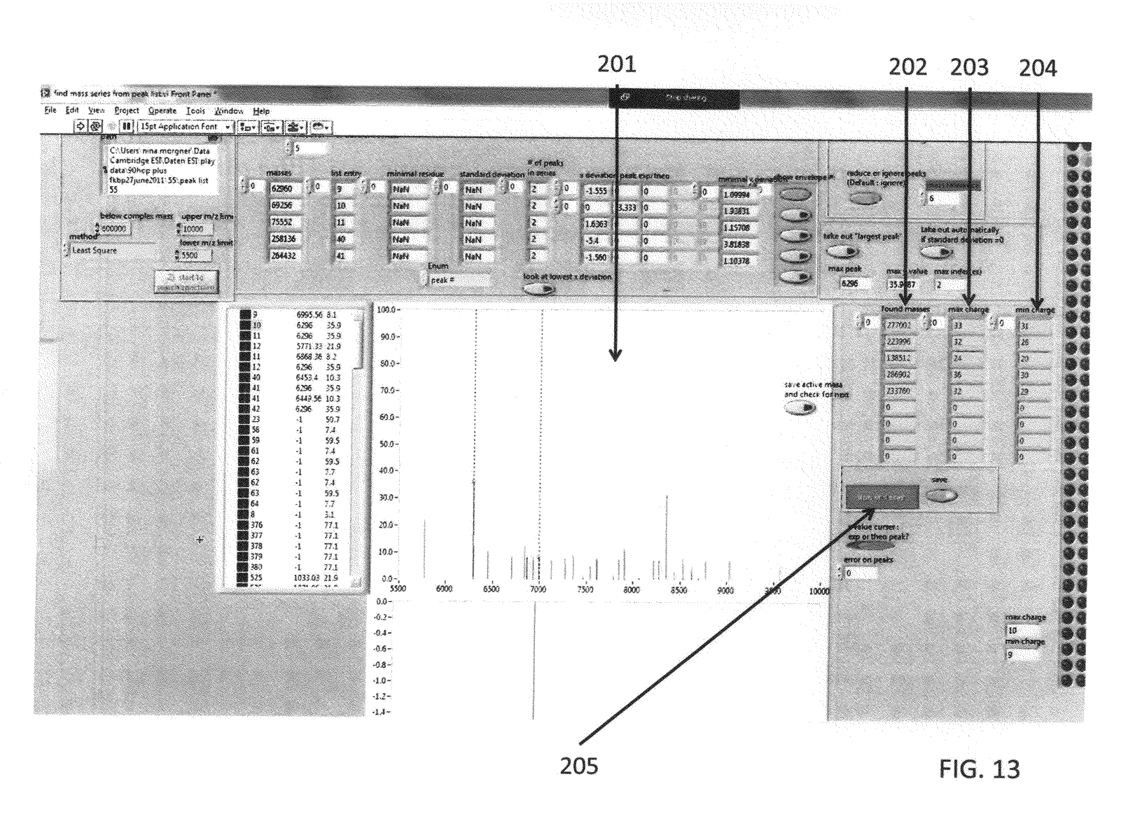

FIG. 11 is a flow chart showing steps in a sub-program for finding mass series automatically. Generally, this sub-program 6000 can be used to determine all the possible peak series from the saved lists of peaks found using the automated peak series find sub-program 4000. The user may load the saved automated peak lists 4011 into the mass series find sub-program 6000. The user can then define 6001 x-axis upper and lower limits. This tells the sub-program 6000 the m/z area in which to look for mass series. For each series the sub-program can select 6002 as candidate peak the peak with the largest y value in the peak list, calculates all the possible mass series that can include the candidate peak from the input lists of found peaks 4011, and then rank the possible mass series according to the number of peaks in the series or deviation of the peaks x/y values from the envelops calculated according to, for example, a Gaussian distribution of the charge states. The user can then evaluate 6003 the most highly ranked found series and determine 6004 whether to accept the found series. If they do not agree 6006 with the found series, the user can view the next possible mass series 6002. This process is repeated until the user is interested 6005 in a found series, at which time the found mass series can be added to a list 6008. The “used” peaks can be subtracted from the peak list 6008. The user then has the option 6011 to look for more mass series using the reduced peak list. If the user is interested 6009 in finding more mass series, the sub-program can be repeated from 6002 using the reduced peak list until the user does not 6013 want to find more mass series. Then the list of found mass series can be saved 6010. This sub-program is also depicted in FIG. 12 and FIG. 13.

FIG. 12 is a screen capture of a user interface for the sub-program for automatically finding mass series, showing user evaluation of a found mass series. The peaks of the series are marked by cursors and a Gaussian envelope curve which is fitted to the peaks is shown to help the user determine if the mass series found is a true mass series, as the peaks that make up a mass series should fall along a Gaussian distribution. To further support this, the user may display x-deviation of the peaks and the theoretical m/z value or their y-deviation from the Gaussian envelope.

FIG. 13 is a screen capture of a user interface for the sub-program for automatically finding mass series. The found mass series are highlighted on the peak lists that are displayed 201. The user can stop the calculation of mass series at any time and save the found mass series by clicking on a stop-and-save icon 205. The results of a found mass series can be displayed, for example shown in the boxes, which are the masses in the series 202, the maximum charge associated with the mass of each peak in the mass series 203, and the minimum charge associated with the mass of each peak in the mass series 204.

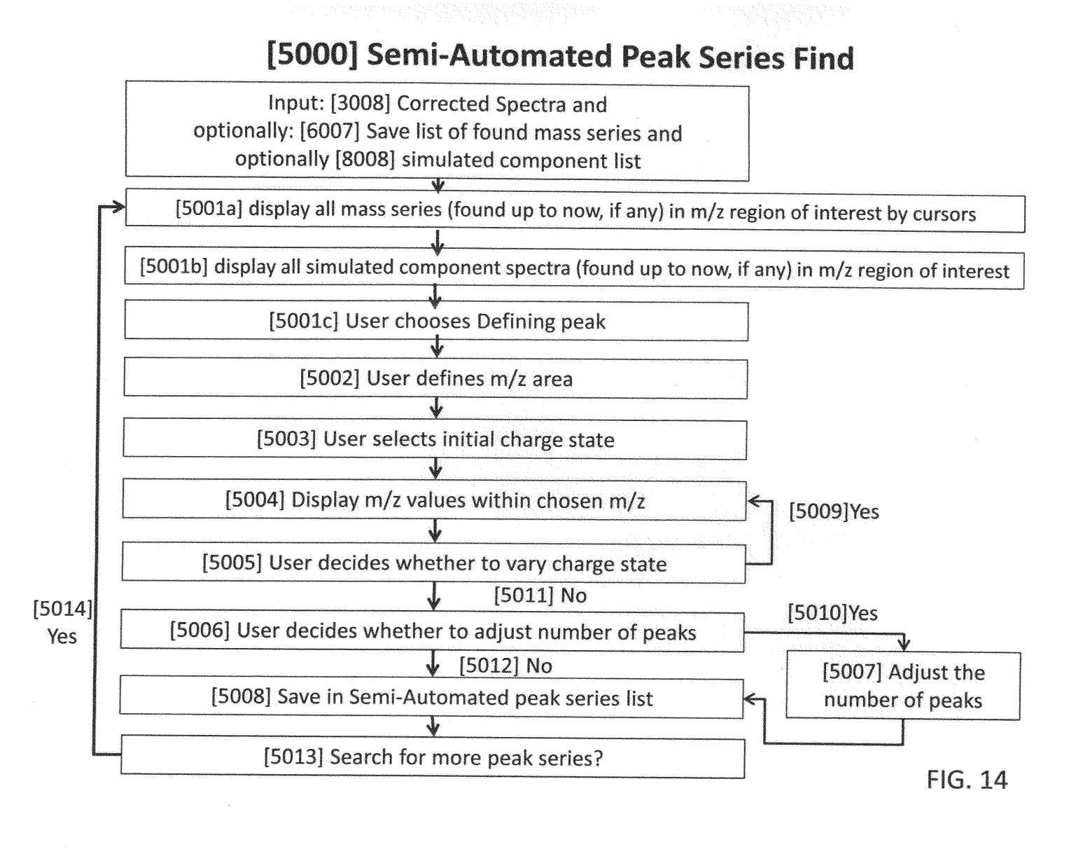

FIG. 14 is a flow chart showing steps in a sub-program 5000 for semi-automatically finding peak series. This program can be applied, if the spectra are not well resolved enough for the automatic peak find program 4000. It is also possible that the peak list 4011 resulting from the automatic peak find sub-program 4000 is incomplete. An incomplete peak list can result in an incomplete peak series list 6010. If the user assumes that more peak series are in the spectrum than found up to now, missing peak series may be found using a semi-automatic peak series find sub-program. In one embodiment smoothed, linearized, background-corrected spectra 3008 and (optionally) peak series lists found 6010 using the automatic mass series find sub-program 6000 can be loaded into this sub-program 5000 and all the found mass series in the m/z region of interest displayed 5001a. If none are found yet (4000 and 6000 not used) the user can start to search for peak series in 3008. If the user has already simulated component spectra 8008 these can be displayed here as well 5001b. Upon receiving its input the user can chooses 5001b a defining peak, which normally may be the largest peak within any particular series, which is not yet part of an assigned mass series. From that chosen 5001c peak, the user can define 5002 a m/z area and select 5003 an initial charge state. The sub-program 5000 can then display 5004 other m/z values in the defined 5002 m/z area, representing m/z values of the mass corresponding to the selected peak and charge, which can be compared with the mass spectrum 3008. Thereafter, the user can decide 5005 whether or not the charge state should be varied. If the user wishes to vary the charge state 5009, then the sub-program 5000 shows 5004 other m/z values in the defined m/z area until the user elects 5011 not to vary the charge state.

Once the user has chosen 5011 not to vary the charge state, the user can determine 5006 whether or not to adjust the number of peaks. If the user opts not 5012 to adjust the number of peaks, the semi-automated peak list can be saved 5008. Conversely, if the user opts 5010 to adjust the number of peaks, he or she can 5007 adjust the number of peaks and save the found series 5008 in the peak list. If the user wants to search for more mass series, this can be done restarting from 5001a.

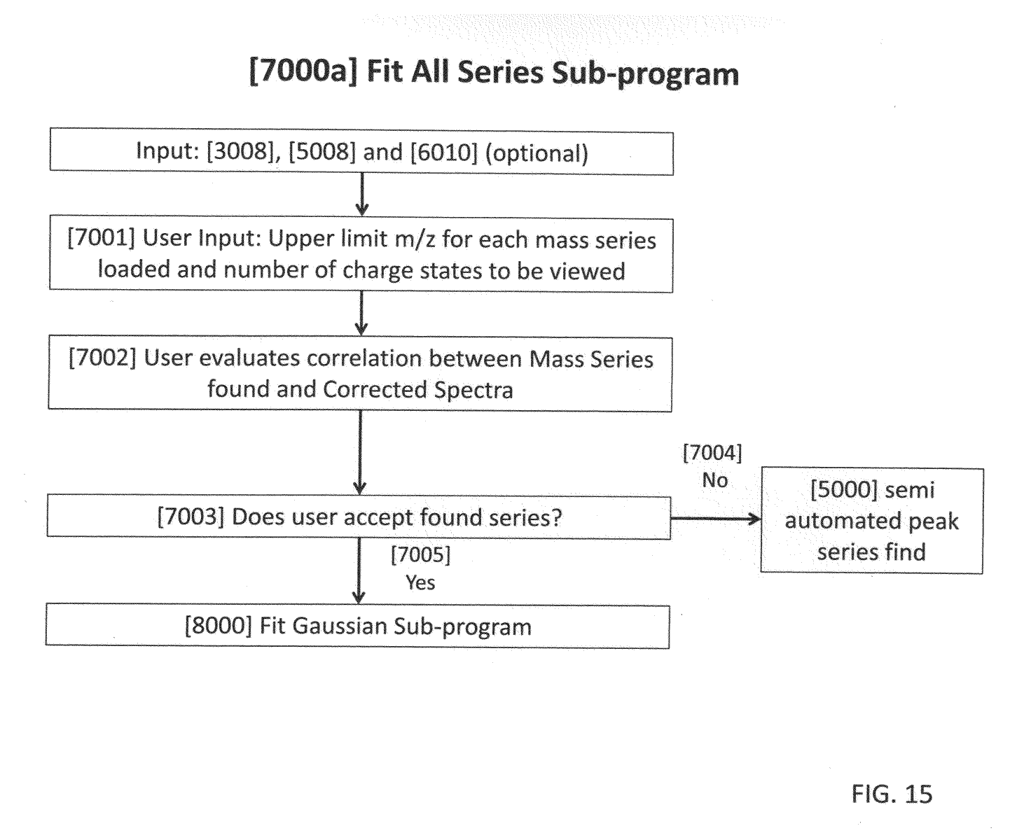

FIG. 15 is a flow chart showing steps in a sub-program for fitting mass series 7000a to an experimental spectrum. The saved semi-automated peak list 5008 and the saved corrected spectra 3008 (experimental spectra) are loaded. Optionally, the user can load the saved [6010] mass series lists determined automatically. The user can define 7001 an upper limit m/z for each mass series displayed and number of charge states to be viewed. The user can then evaluate 7002, by visual inspection, the correlation between the mass series and the corrected spectra 3008 (experimental spectra). The user can then determine 7003 whether or not to accept the found mass series. If the user accepts 7005 the found mass series, then the found mass series may be simulated using a sub-program 8000 to fit Gaussians. If the user does not accept 7004 the found mass series then the user may search for more mass series using the semi-automated peak series find 5000.

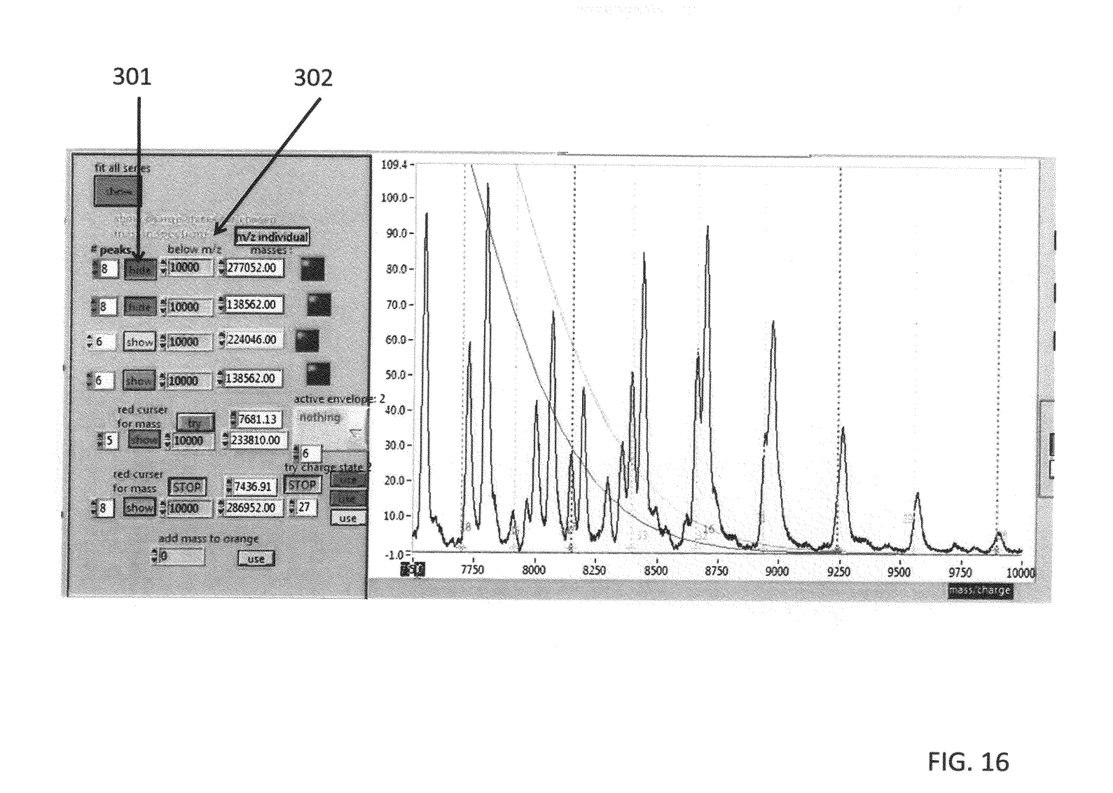

FIG. 16 is a screen capture showing a user interface for the sub-program for fitting mass series to an experimental spectrum described in FIG. 15. Found mass series may be loaded into the program and each mass series loaded represented differently (e.g., by a different color) 301. The user may enter 302 the maximum m/z (x-axis value) to define the area of the theoretical peak distribution (in this screenshot: green cursers) to be displayed. Alternatively, the user searches for masses using sub-program 5000 and displays them. The black spectrum shown is the corrected spectrum (experimental spectrum) and the peaks in each mass series are represented by dotted vertical lines in colors correspond to the mass series that they belong to. In other words, all the peaks belonging to the mass series represented by a color, for example by green, are shown by a vertical green dotted line. This representation allows the user to see if the dotted vertical lines correlate with peaks along the experimental spectrum.

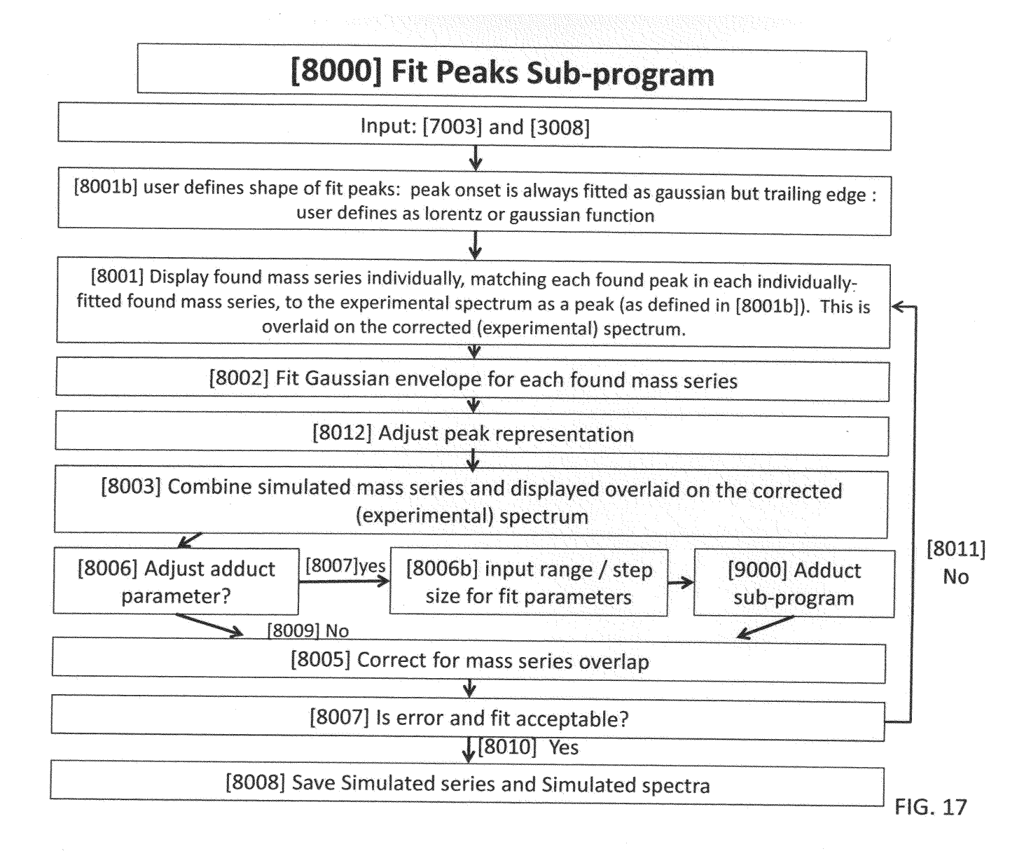

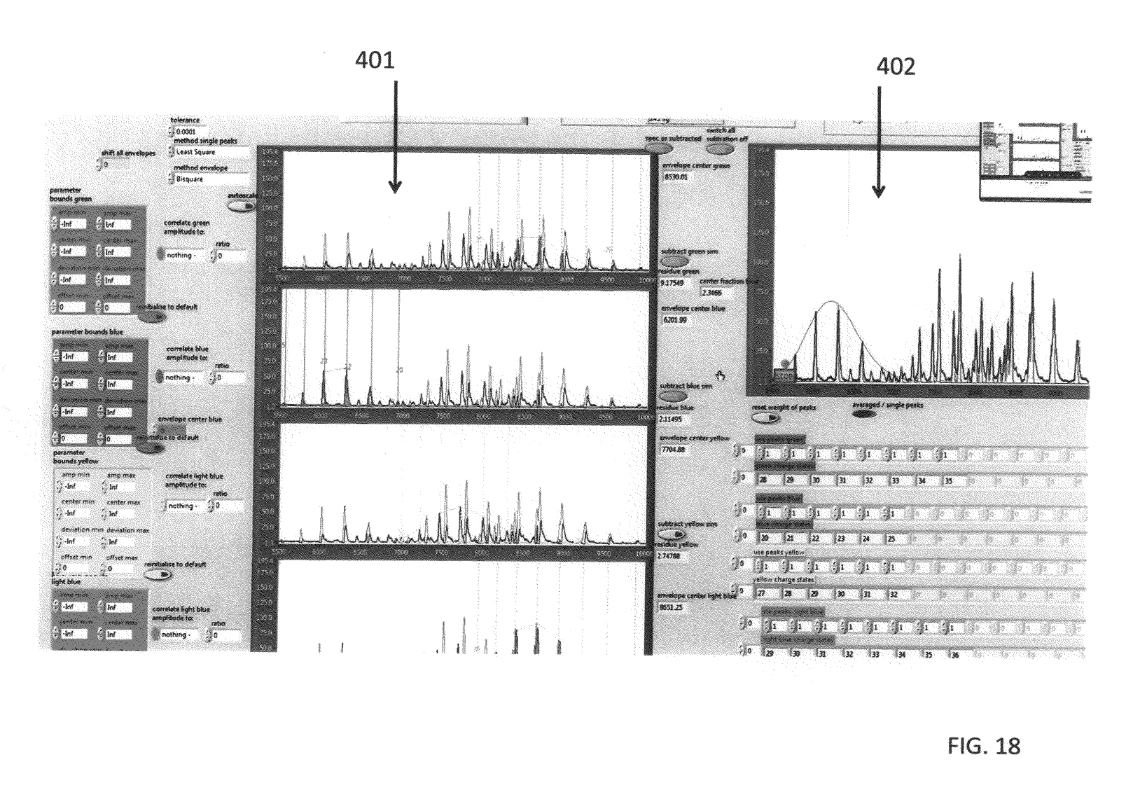

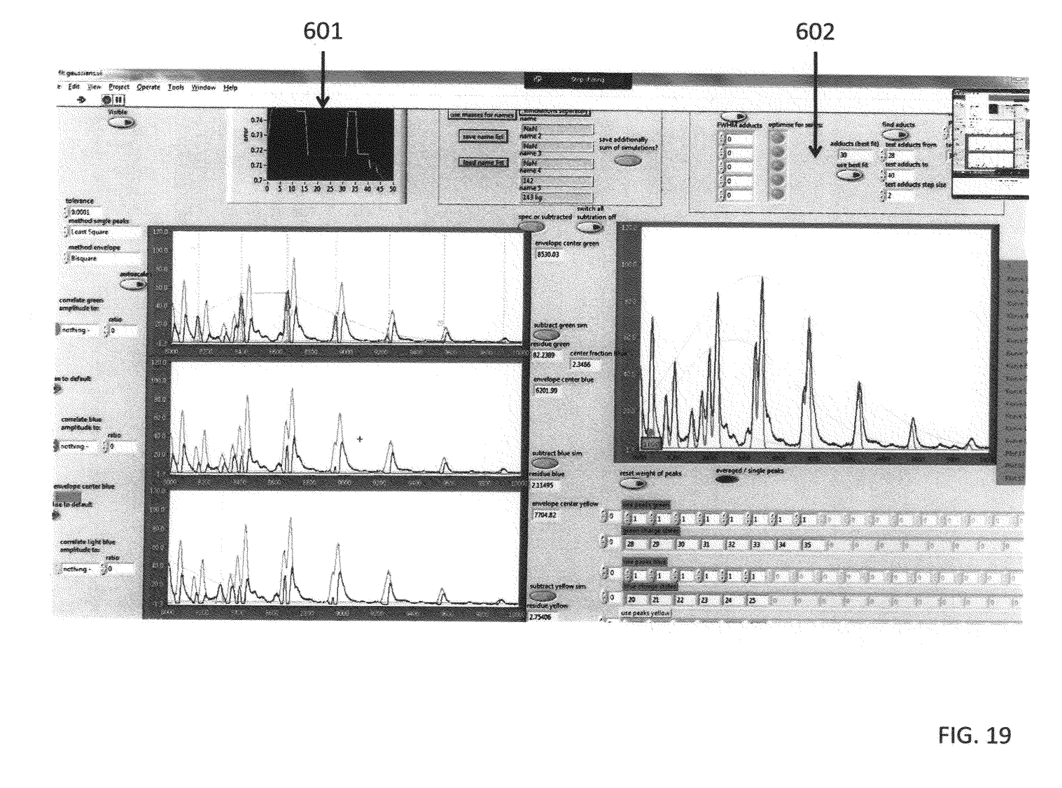

FIG. 17 is a flow chart showing steps in a sub-program 8000 for fitting peak representations to found peaks in mass series to generate simulated series. Mass series that are determined by the user to correlate to the experimental spectrum can then be loaded into a Fit Peaks Sub-program 8000 to simulate the peaks and mass series using, for example, Gaussian distributions. Up until this point peaks are represented by cursors. The sub-program 8000 thus fits 8001 each found series individually with each found peak in each found mass series being fitted individually rather than a vertical line. The peaks onset can be fitted as gaussians and the trailing edge either as Gaussian or as Lorentz curve—as defined [8001b] by the user. The fitted mass series are displayed overlaid on the corrected (experimental) spectrum. This is shown in FIG. 18. The sub-program 8000 can then fit a Gaussian envelope 8002 for each mass series to encompass all the fitted peaks, with the Gaussian over the charge states, not the m/z scale.

Next, the peak representation can be adjusted 8012. To adjust the peak representation, the molecule's mass can be calculated, for example as an average from the masses determined by multiplying each fitted peak center (m/z value) by the peaks charge. Every peak in the series can be simulated by a Gaussian (or trailing edge lorentzian as described above) with the center being calculated according to the thus determined mass, the peak width as the average of the peak fits and the peak height according to the envelope. All the simulated mass series can be combined and displayed overlaid on the corrected (experimental) spectrum 8003. Possible peak overlap in the mass series can be corrected for in 8000 by repeating the described fit routine 8001-8003. However, the input is all other simulated peak series subtracted from 3008 and not the experimental spectrum 3008. The deviation between the simulated combined spectrum (i.e., the spectrum generated when all loaded found mass series are combined) and the experimental spectrum can be displayed. The user can stop the mentioned fit procedure when this deviation is no longer minimized by further fit rounds and thus all the possible peak overlap in the mass series has been corrected for.

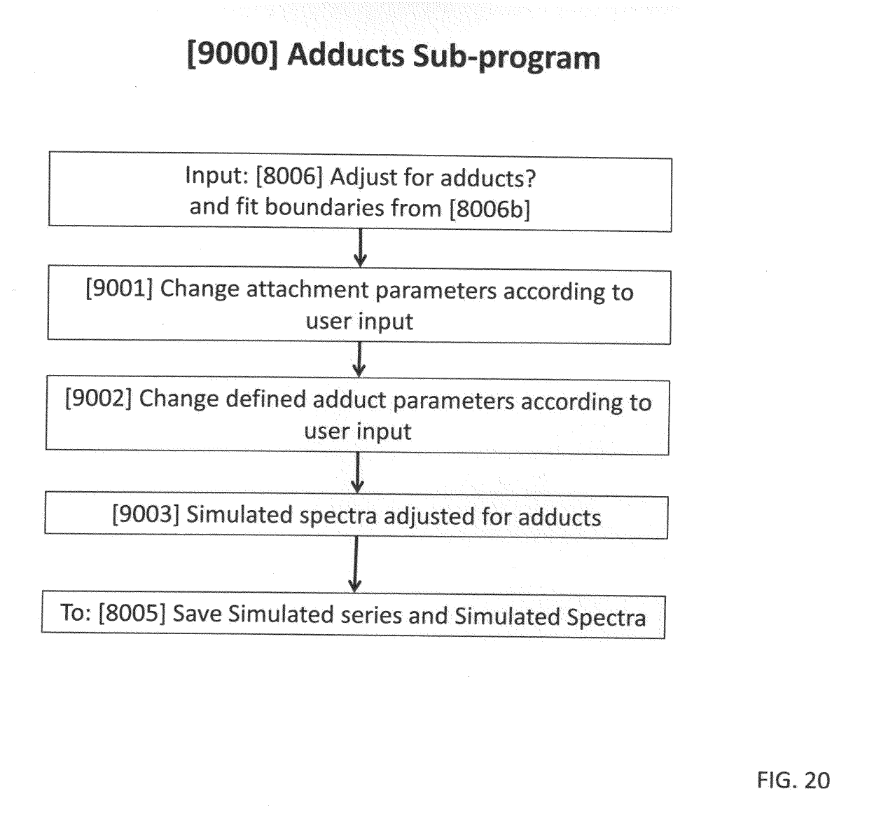

As an optional step the user can determine 8006 if the peaks need to be adjusted for adducts. These adducts can be a distribution of small adducts (for example, water, buffer, salt molecules), which may broaden the peaks. We also refer to these adducts as attachments. It is possible that multiple molecules (the number of which may be determined by the user) of defined mass (added by the user, for example, detergents) attach, which can be resolved in the spectra. We refer to these as defined adducts. Both attachments and defined adducts can appear at the same time and may be fitted according to one fit parameter each (broadening of the trailing edge and height of the defined adduct signal in comparison with the peak that does not contain the additional mass). If adducts are present 8010, the spectrum can be corrected for adducts using an adduct sub-program 9000, which can be accessed from the Fit Peaks sub-program screen and runs as a sub-routine in the fitting process, if the user activates it. For this the user may add [8006b] upper and lower limit and step size for the fit of the two attachment parameters. In case of defined adducts the user can enter the mass of the adduct and the maximum number of adducts (too many is no problem). This is shown in FIG. 19. The adduct parameters can be varied according to the user's input and the fit routine 8001-8003 repeated to optimize the overall fit. The program calculates the deviation of the fit with the experimental spectrum, and determines for which adduct parameter this deviation is smallest. The adduct sub-program is described in greater detail with reference to FIG. 20. If no adjustment for adducts is needed 8011, or sub-program 9000 has been used to adjust for adducts, then the spectra are corrected for mass series overlap 8005. To correct for mass series overlap, for every series to be fitted, the experimental spectrum is replaced by all other fitted series subtracted from the spectrum. The user can then analyze the resulting simulated mass series and simulated spectra to determine if the error and fit of the simulated spectra and simulated series are acceptable. If the error and fit of the simulated series and simulated spectra are acceptable 8010, then simulated mass series and simulated spectrum are saved 8008. If the error and fit of the simulated series and simulated spectra are not acceptable 8011, then the sub-program is repeated beginning at 8001.

FIG. 18 is a screen capture showing a user interface for the sub-program 8000 for fitting Gaussians to found peaks and mass series described in FIG. 17. Each simulated mass series is represented differently (e.g., by a different color) and is displayed against the experimental spectrum (shown in red) individually 401. In the later fit rounds the experimental spectrum is used for the fit but the experimental spectrum from which all other simulated spectra are subtracted (shown in black in 401). The combined simulated spectra are also displayed with the mass series Gaussian envelopes and experimental spectrum 402. The combined spectrum is shown in red and each Gaussian envelope is overlaid on the combined simulated spectrum and is shown in the color matching the color of the mass series it represents.

FIG. 19 is a screen capture showing the sub-program for fitting Gaussians to found peaks and mass series described in FIG. 17 showing a correction for peak overlap function. The error of the simulation is shown 601. The adducts sub-program 9000 can also be accessed 602 from this sub-program 8000.

FIG. 20 is a flow chart showing steps in a sub-program 9000 for adjusting for adducts. Adducts may form due to experimental conditions while obtaining a raw mass spectrum which leads to trailing edges on the high mass side of mass peaks. In the case of peak overlap, a peak sitting in the trailing edge of a different peak would not be represented correctly when fitted by a Gaussian. The adduct sub-program 9000 adds mass adducts to all the peaks in a spectra. Since all species in a sample undergo the same conditions inside the mass spectrometer, the default setting is that the adduct parameter is kept the same for all peaks. Adjusting for adducts may be important when quantitative analyses are to be performed.

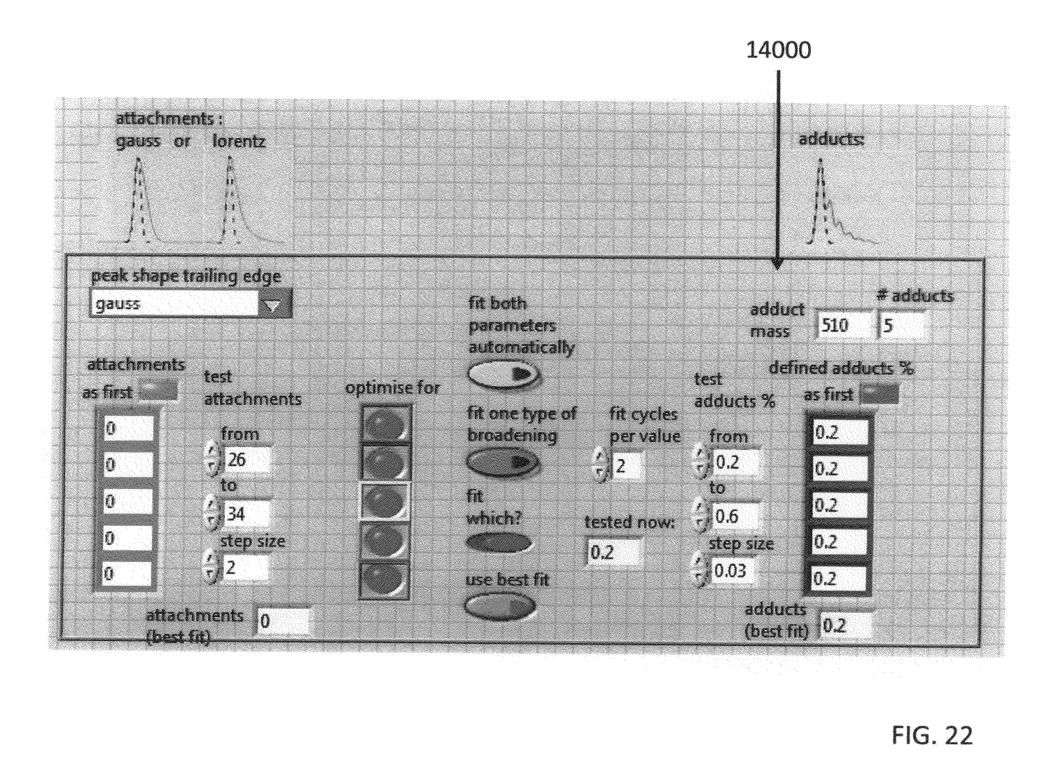

Sub-program 9000 is accessed from the Fit Gaussian sub-program 8000 when the user decides to 8006 adjust for adducts. The user inputs 8006b test adduct parameters. Parameters to be entered are shown in greater detail in FIG. 22. In each fit round the sub-program changes the adduct parameters as requested. The simulated spectra may then be adjusted 9003 according to the adduct parameters and the simulated series and spectra returned to 8005 in the 8000 fit routine to correct for mass series overlap and determine the deviation of simulated and experimental spectrum. The best parameters for adduct (smallest deviation) may be determined and used for the fit.

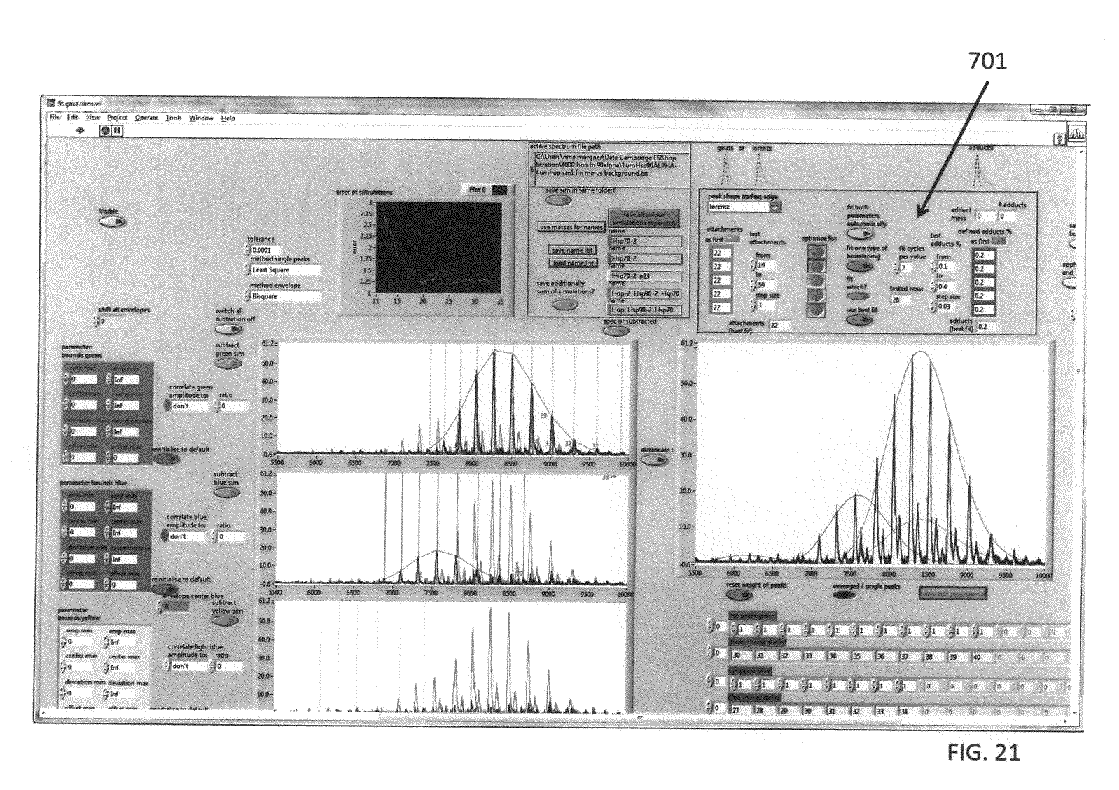

FIG. 21 is a screen capture showing a user interface for the sub-program for adjusting for adducts described in FIG. 20. This screen capture shows the adducts sub-program 701 as viewed from the user interface of the Fit Gaussian sub-program.

FIG. 22 is a zoomed-in screen capture showing a user interface for the sub-program for adjusting for adducts described in FIG. 20. The user may enter parameters 14000 to test for the amount of adduct to add and then run the sub-program 9000.

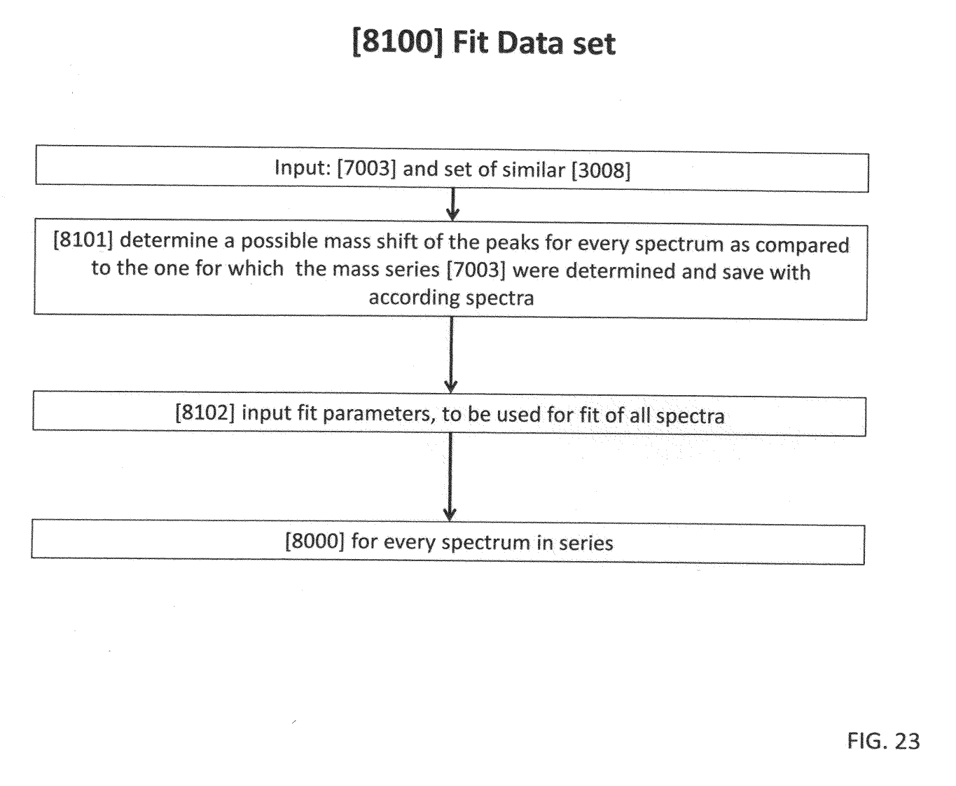

FIG. 23 is a flow chart showing steps in a sub-program that can be used to fit a set of spectra with the same complexes and same boundaries for fit parameters. The user can select a set of spectra 3008 (for example same complexes at different concentrations or time points) and determine potential small shifts in the peak positions, which would lead the masses 7003 determined for one of the spectra not to fit exactly the other spectra. This would cause the cursors that mark the m/z value for each peak not to be in the middle of each peak. The user [8101] determines the shift necessary to correct this for each spectrum. They can then be saved with each spectrum. The user can then enter a sub-program [8102] and input the fit parameter boundaries to be used for the fit of all spectra. The program then calls the peak fit program 8000 for each spectrum.

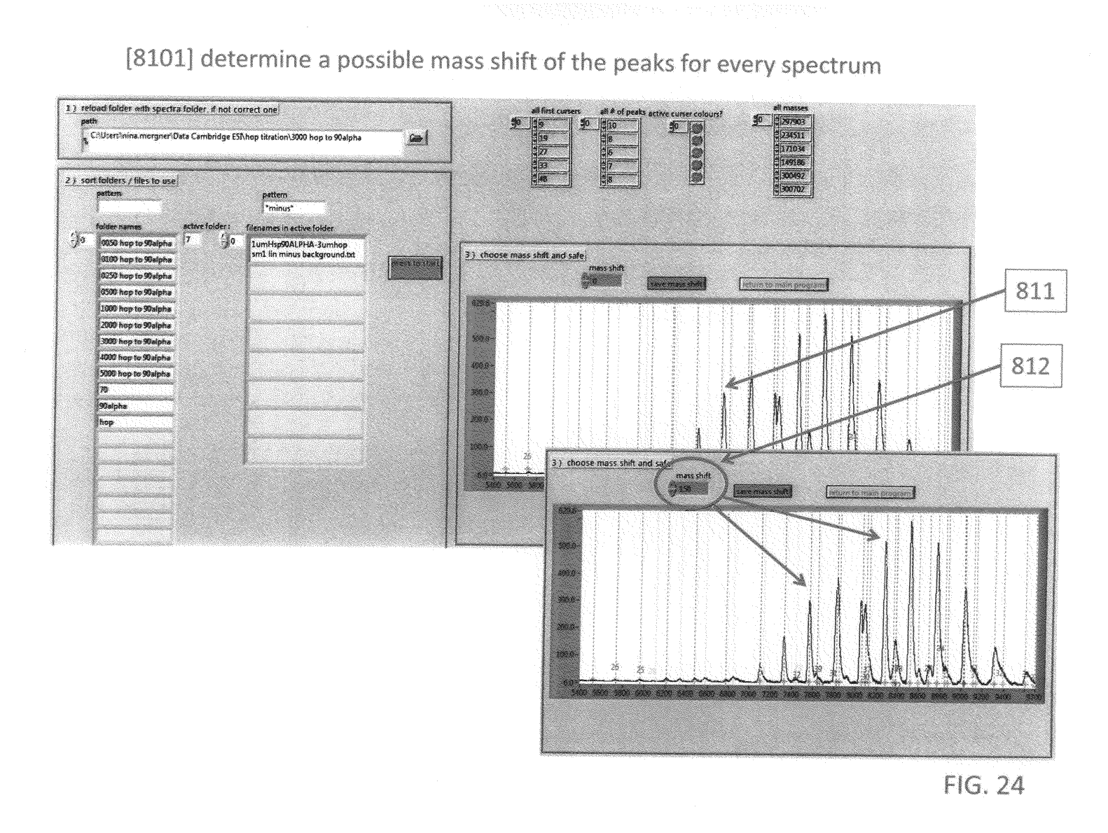

FIG. 24 is a screen capture showing how a mass shift needed to use the same peak series masses for the fit of a set of spectra may be determined. If there is a shift causing the cursors not to lie in the peak centers 811 the user inputs a mass shift, which corrects that 812 and saves the result.

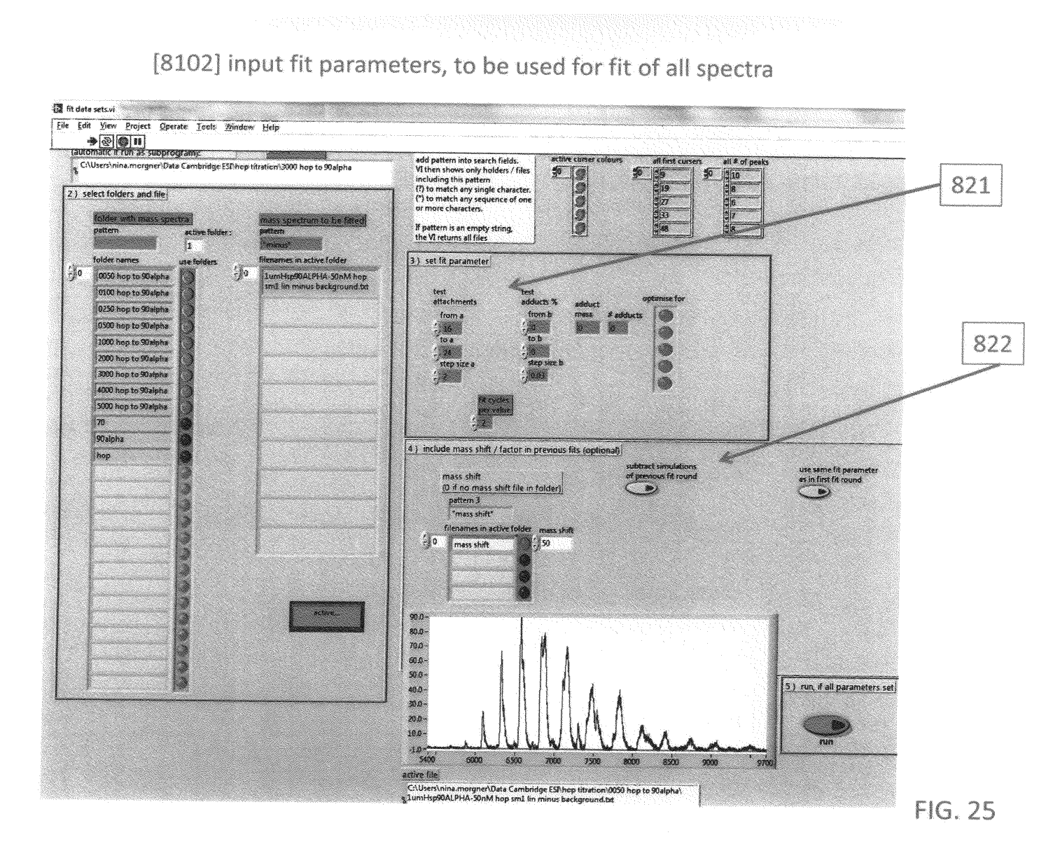

FIG. 25 is a screen capture of a program in which the user can input the fit parameter boundaries for the fit of each spectrum of a series. These parameters 821 are for attachments and defined adducts for the fit of the first 5 mass series. In case additional mass series have to be fitted the program allows to read out and use 822 previous fit parameters and simulations to be subtracted from the experimental spectrum before the fit of the additional series.

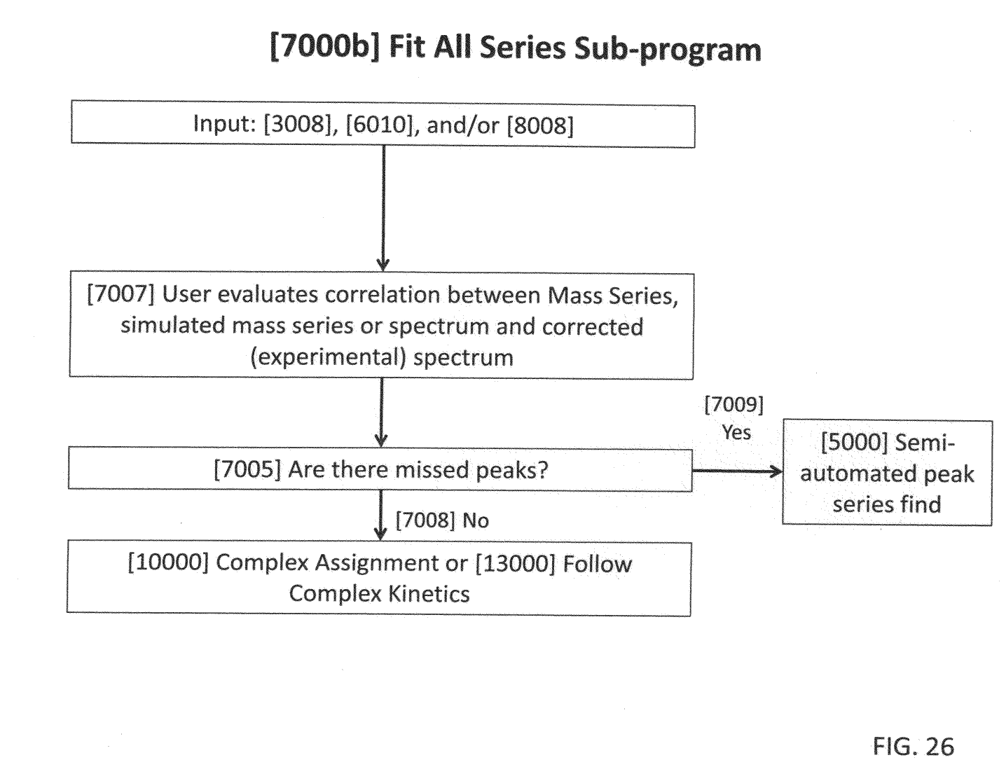

FIG. 26 is a flow chart showing steps in a sub-program 7000b for fitting mass series, a simulated series or spectrum, an experimental spectrum and identifying and accounting for missed peaks. In addition to the corrected spectrum 3008 (experimental spectrum), a found mass series 6010, and/or a simulated series or spectrum 8008 are loaded. The user can then evaluate 7007 the correlation between the mass series, simulated mass series or spectrum, and corrected (experimental) spectrum. The user can then determine upon visual inspection if 7005 there are any missed peaks. If there are missed peaks 7009, then the user can enter into 5000 to find the mass series the missed peaks belong to. If no peaks are missed 7008 then the simulated series and spectrum are ready to be used for 10000 assigning complexes or for 13000 following complex kinetics.

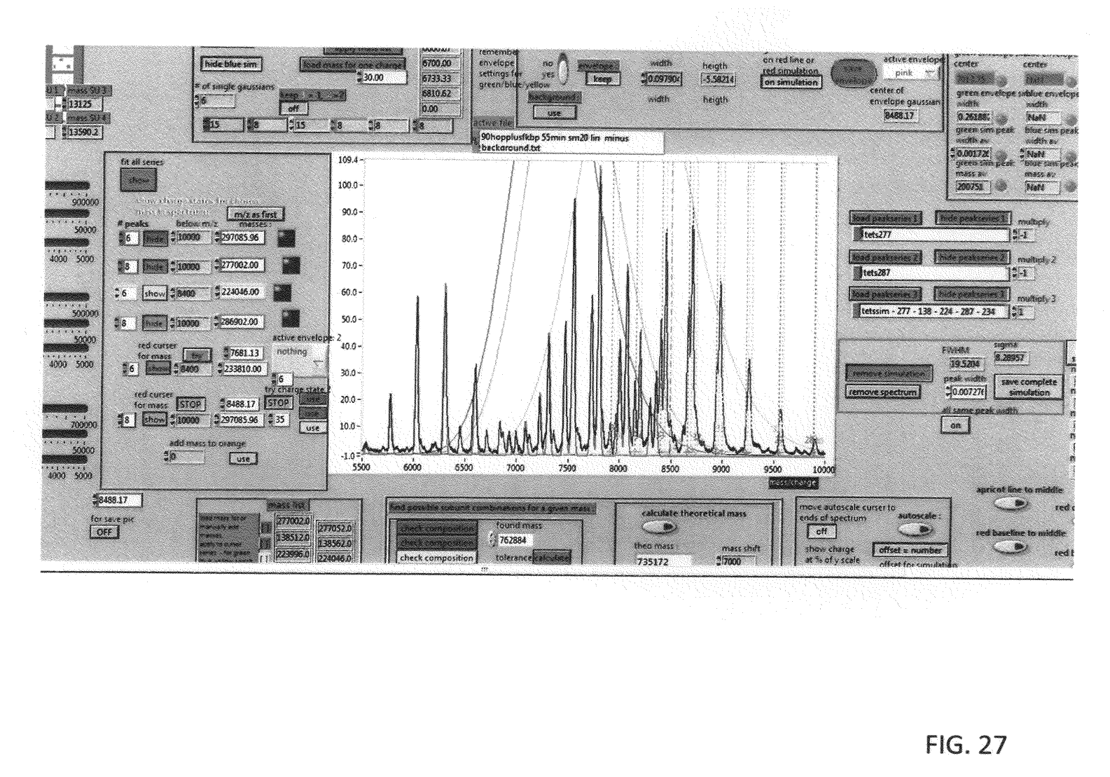

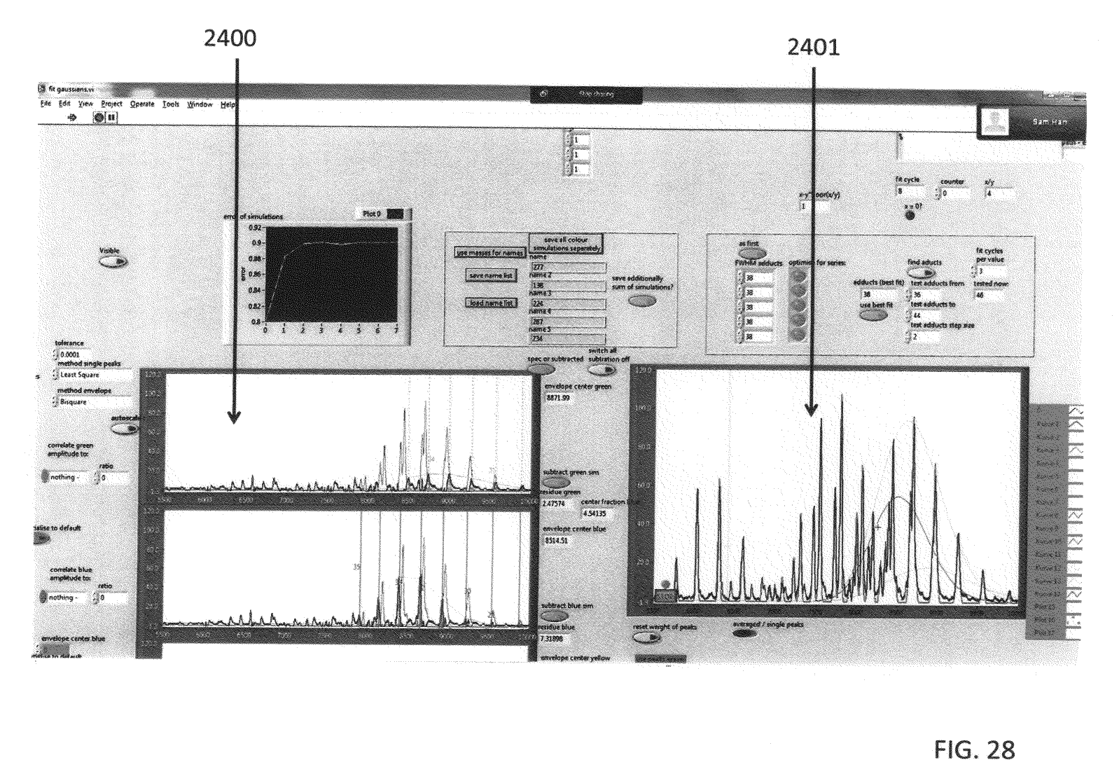

FIG. 27 is a screen capture showing a user interface for the sub-program for fitting mass series. A partially simulated spectrum is shown in red (from 8008), an experimental spectrum is shown in black and not yet accounted for peaks (missed peaks as described in FIG. 26) are identified. The peak series to which the missed peaks belong were determined in sub-program 5000 and those mass series are represented by green, dark blue and light blue cursers, ready to be fitted in sub-program 8000.

FIG. 28 is a screen capture showing the use of the Fit Gaussian sub-program described in FIG. 17 and FIG. 26 to fit additional peak series, indicated by cursors in FIG. 27. The previously fitted peak series (red in FIG. 23) are subtracted from the spectrum to be fitted 2400. 2401 shows the experimental spectrum and the simulation including the previous fits, that were subtracted in 2400.

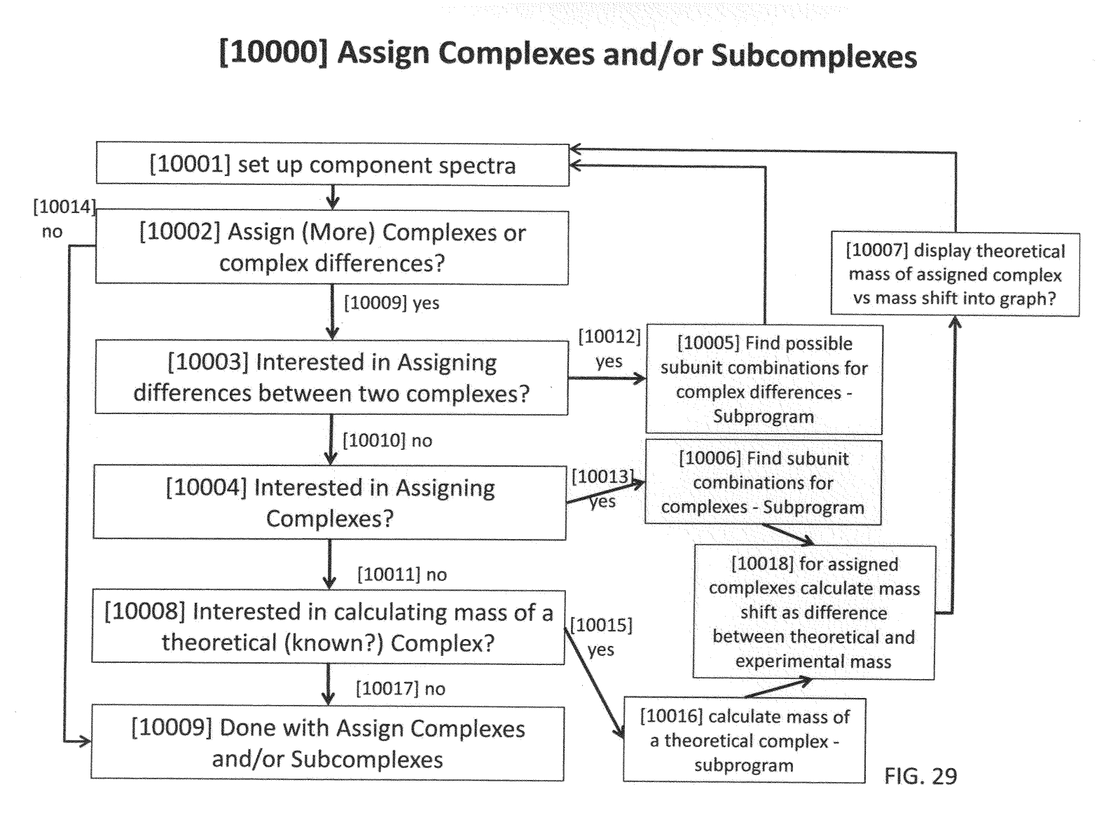

FIG. 29 is a flow chart showing steps and sub-programs that can be used to assign complexes and/or sub-complexes 10000 to a found mass series. The portion of the program that assigns complexes and/or sub-complexes relies on the individual use of several sub-programs including the set up component spectra sub-program 10001, the find complex sub-program 10006 and the find possible subunit combinations sub-program 10005. Although one order is shown in FIG. 29 and described here, this is only one embodiment. The sub-programs can be used in any order to analyze the simulated spectra 8007. The order is dependent on the needs of the user.

In this embodiment, one experimental spectrum and all simulated component spectra can be loaded into the component spectra sub-program 10001. The user can then determine how to analyze the component spectra based on the needs of the user. The next several sub-routines using several sub-programs allow for investigation of different questions, followed by returning to the main program. Typically, after setting up the component spectra 10001, the user can then determine whether to assign (more) complexes or complex differences (solution or CID) 10002. If the user is not interested 10014 in assigning more complexes or complex differences 10002, then the user is 10008 done with assigning complexes and/or sub-complexes.

Conversely, if the user is 10009 interested in assigning more complexes or complex differences 10002, the user next decides whether the user is interested in assigning the differences between two selected complexes (simulated components) 10003. This can be used to determine if complexes emerge from each other via loss of sub-units in solution or gas phase. These findings can be used as restraints in the later assignment process. If the user is interested 10012, the user can proceed by using the find possible sub-unit combination sub-program 10005. Once possible sub-unit combinations are found, the process can be continued with the set up component spectra sub-program 10001.

If the user is not 10010 interested in assigning differences between two complexes 10003, the user can then determine whether the user is interested in assigning complexes to the found mass series 10004. Typically, if the user is 10013 interested in assigning complexes, then the find complex sub-program 10006 is used. The user has the option 10008 to calculate the mass of a theoretical complex (for comparison with experimental masses). If the user chooses to do so 10015, the user can use sub-program 10016. For complexes that were assigned to a specific experimental mass via 10006 or 10016 the difference between experimental and theoretical mass (the mass shift), stemming from attachments can be calculated 10018. The user has the option to display the mass shifts for all up to then assigned complexes 10007, which can help further assignment processes. The user returns to 10001 until the user no longer 10008 wants to assign more complexes.

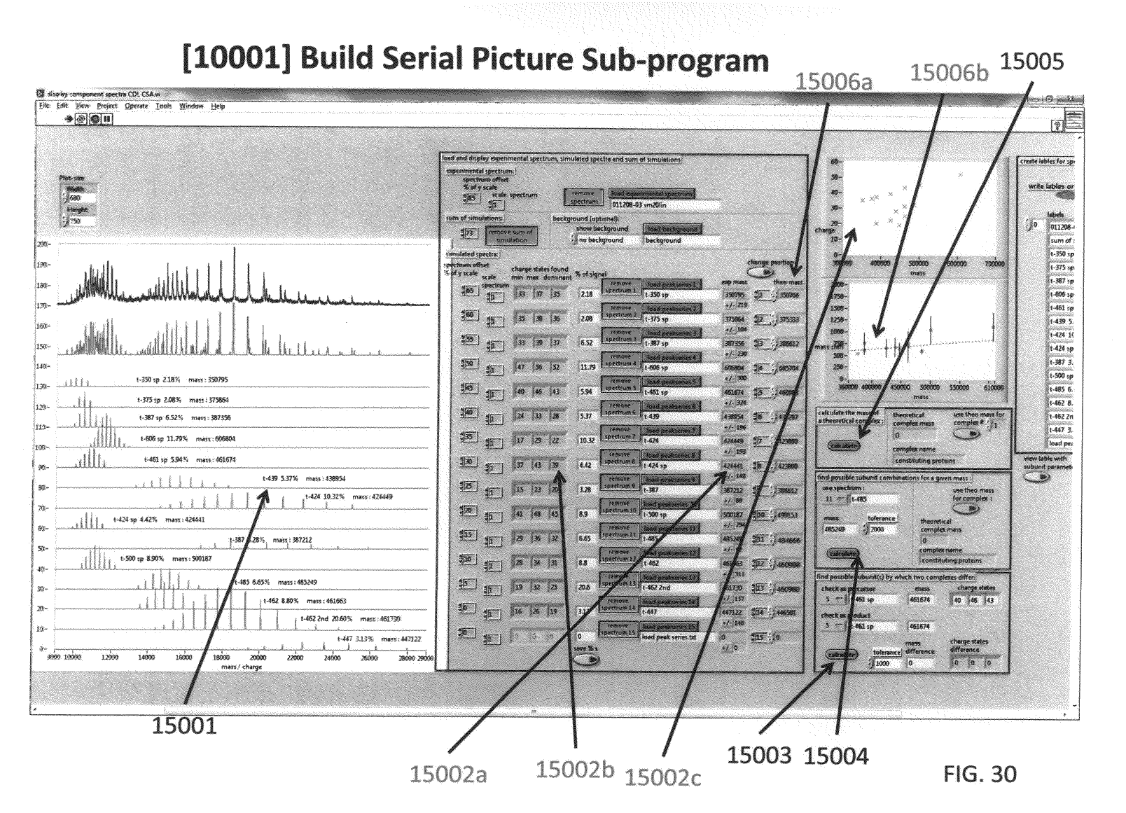

FIG. 30 shows a screen capture of results from a set up component spectra sub-program 10001 that can be used to assign complexes and/or sub-complexes to a found mass series. Corrected spectra 3008, simulated spectra and series 8007 can be loaded and displayed 15001. The complex mass and error 15002a and dominant charge state of the complex 15002b are also displayed and plotted 15002c. These graphs can be used to identify CID and solution complexes. To determine relationships between complexes the user can enter into sub-program 10005 via interface 15003. To assign complexes the user can enter into sub-program 10006 via interface 15004. To calculate the theoretical mass of a complex (known) the user can enter into sub-program 10016 via interface 15005. Theoretical masses of assigned complexes from 10005 and 10006 can be shown 15006a and the mass shifts vs. mass 10007 displayed 15006b.

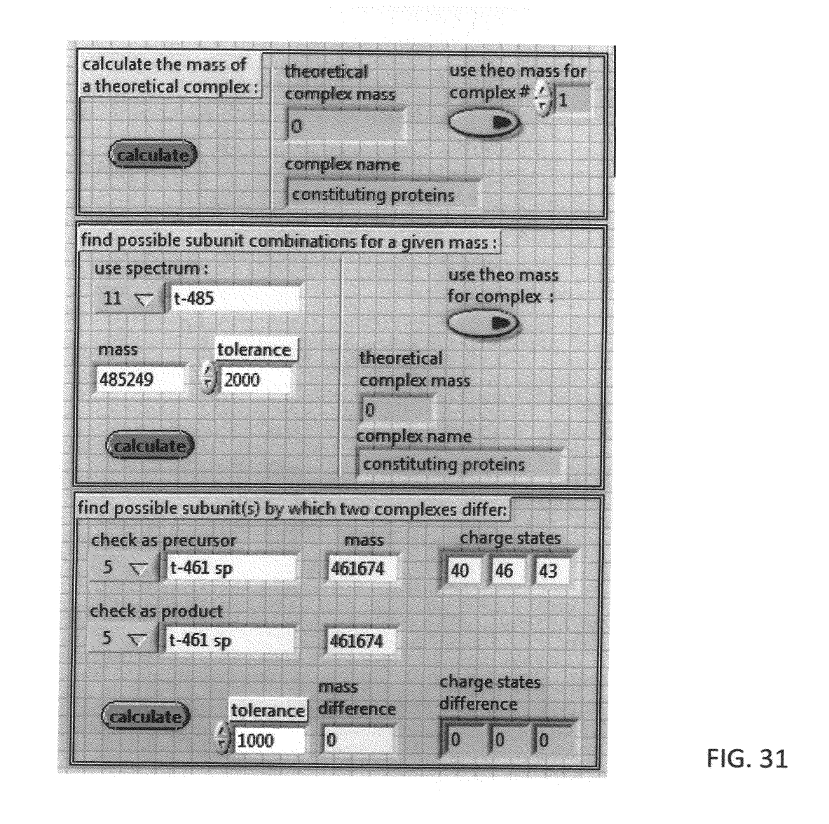

FIG. 31 shows a portion of the user interface of FIG. 30 in greater detail.

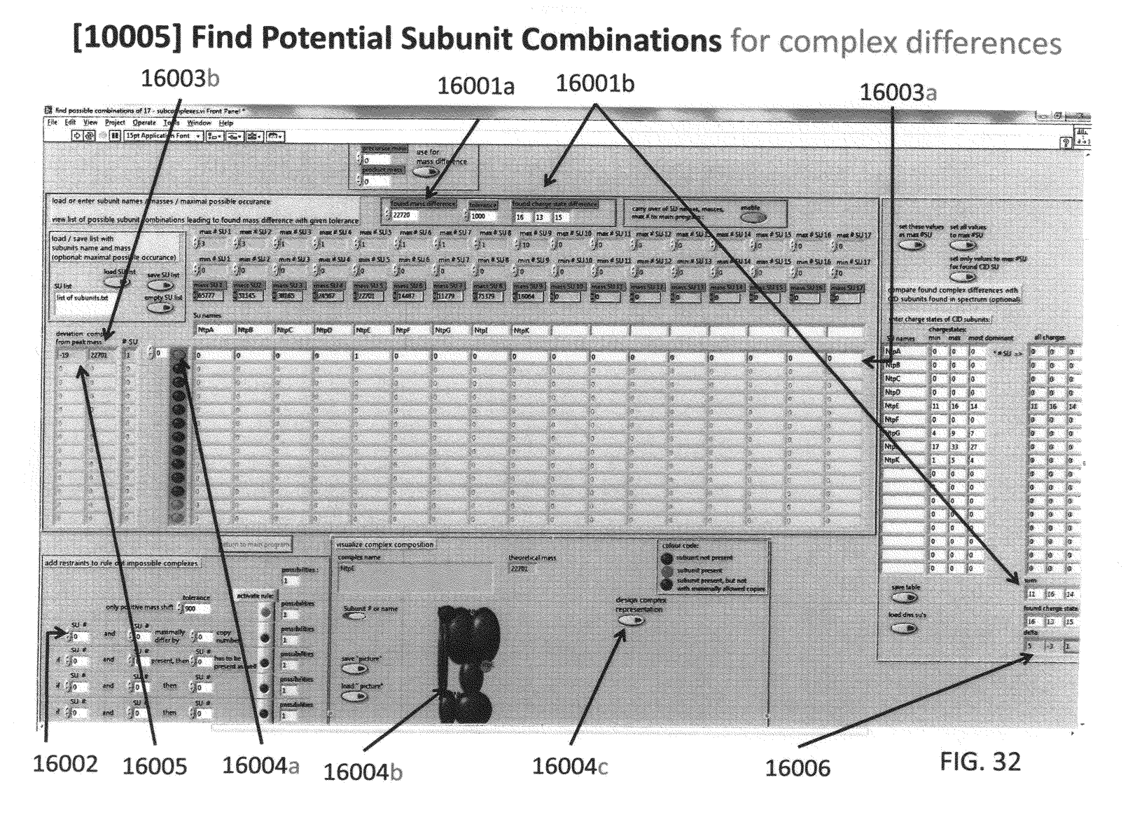

FIG. 32 shows a screen capture of a user interface for the find possible sub-unit combinations sub-program that can be used to assign sub-units and charge states by which two found mass series differ. Two mass series can be selected 15003 (FIG. 26) and mass and charge differences transferred into this sub program 16001a and 16001b. (Or can be entered here, if this program is used alone.) The user may enter constraints 16002 based on known information such as known interactions, stoichiometries, or biologic impossibilities. The sub-program calculates the possible sub-unit(s), which can explain the difference and lists them 16003a as well as the theoretical mass of the complex 16003b. The user can select to display 16004a the finding in a color coded complex schematic 16004b, that can be set up through a sub-routine, that can be called from 16004c. The deviation in mass 16005 and charge 16006 of theoretical complex and experimental finding for the selected solution is displayed. The user may save or lock in an assignment at any time, thus adding another constraint for the sub-program to consider.

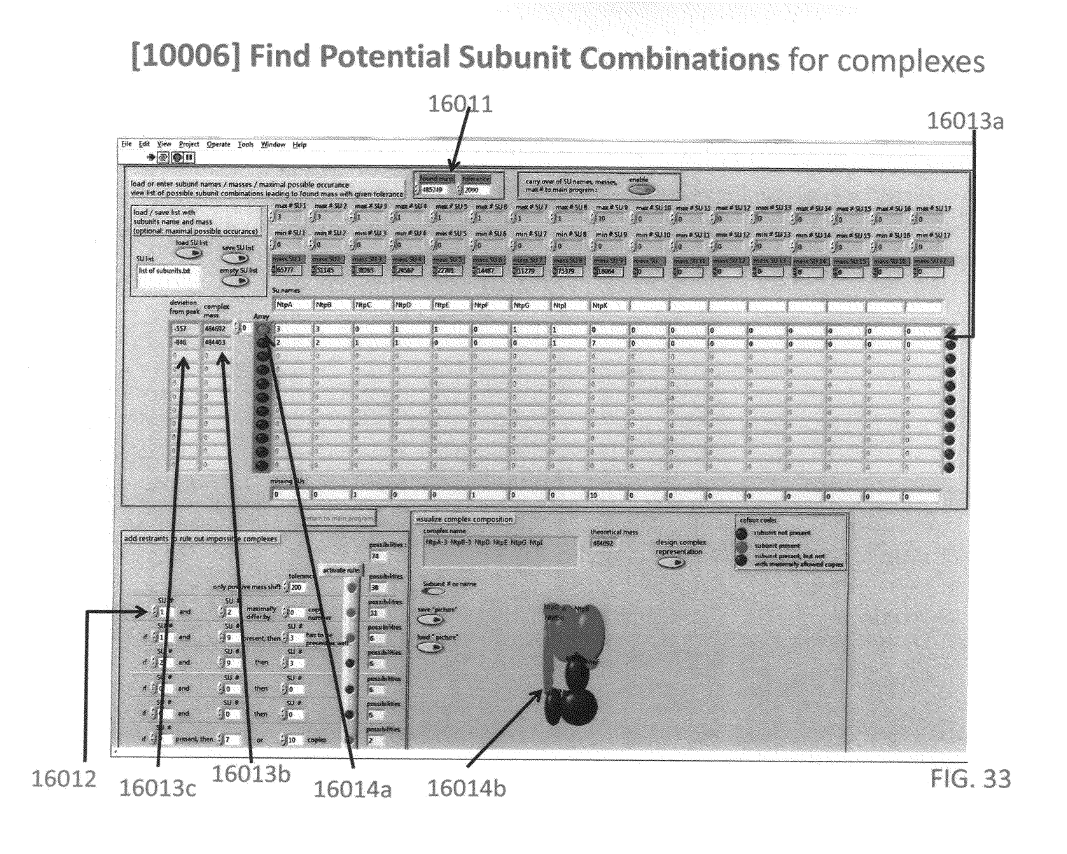

FIG. 33 shows a screen capture of a user interface that can be used to assign sub-unit combinations to complex masses. The complex mass and tolerance (default 2000 Da) is transferred 16011 into this sub-program. (Or can be entered here, if this program is used alone.) The user may enter constraints 16012 based on known information such as known interactions, stoichiometries, or biologic impossibilities. The sub-program can calculate the possible sub-unit(s), whose combination would lead to the found mass and lists them 16013a, as well as the theoretical mass 16013b and the deviation between this mass and the experimental complex mass 16013c. The user can select to display 16014a the finding in a color coded complex schematic 16014b

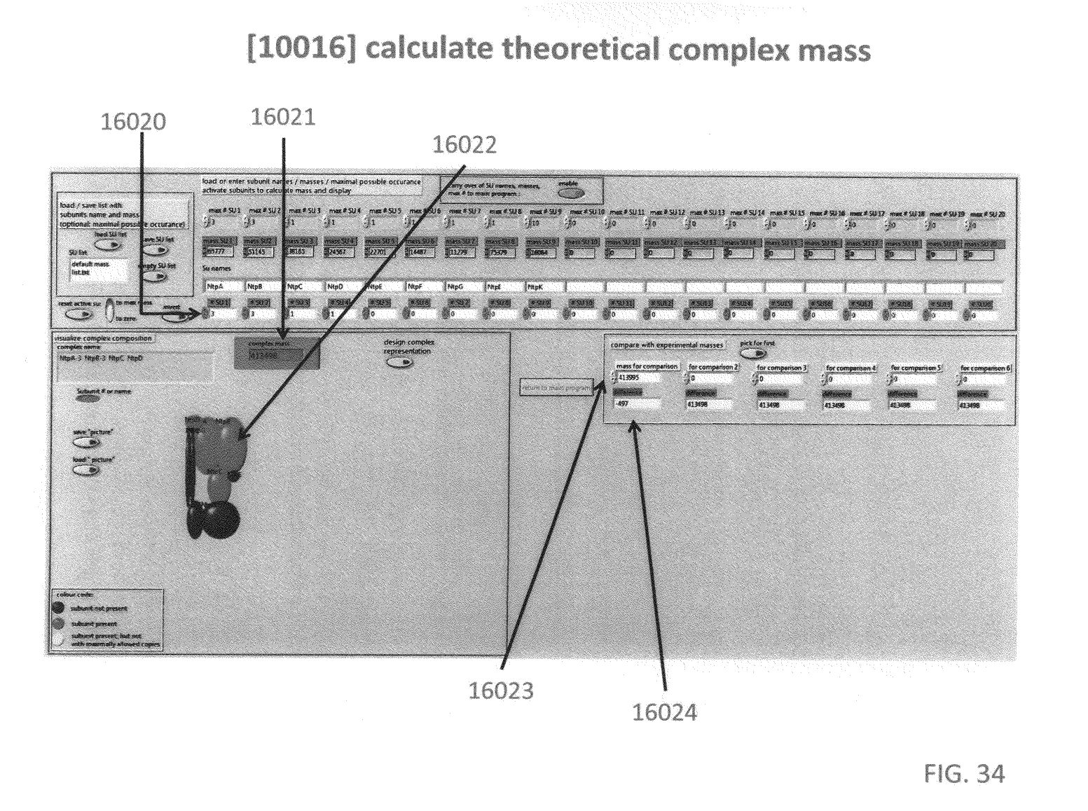

FIG. 34 shows a screen capture of a user interface that can be used to calculate the mass of a theoretical complex (a certain subunit combination), by entering the copy number for each subunit 16020. The mass of this complex 16021 and a color coded schematic 16022 are shown. The user can enter a (experimental) mass 16023 and the difference between this and the theoretical mass is displayed 16024.

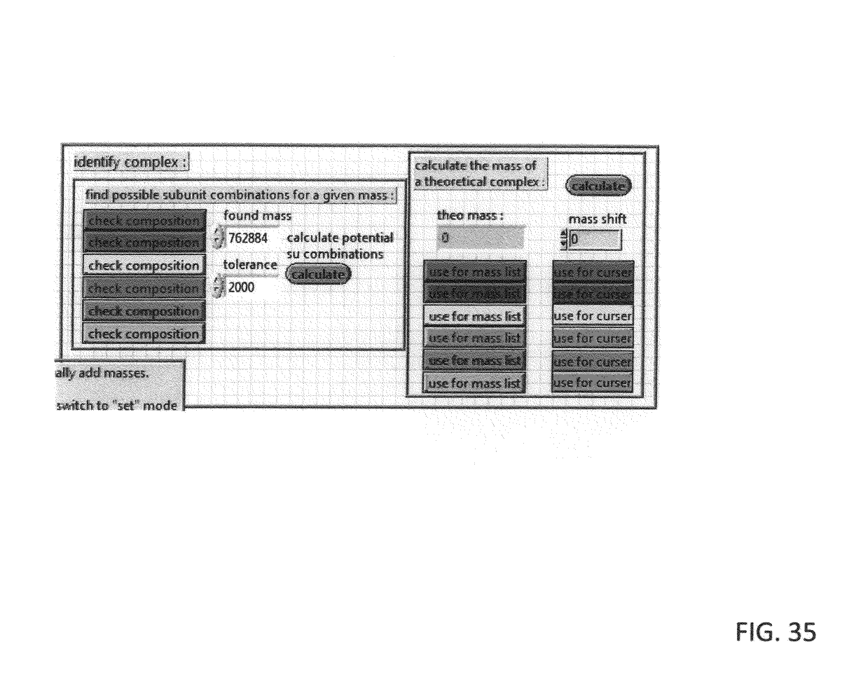

FIG. 35 shows a portion of the main call screen of FIG. 23, showing a user interface to alternatively access from the main program the subprograms to find possible subunit combinations 10006 (FIG. 25) and to calculate the theoretical mass of a complex 10016 (FIG. 25).

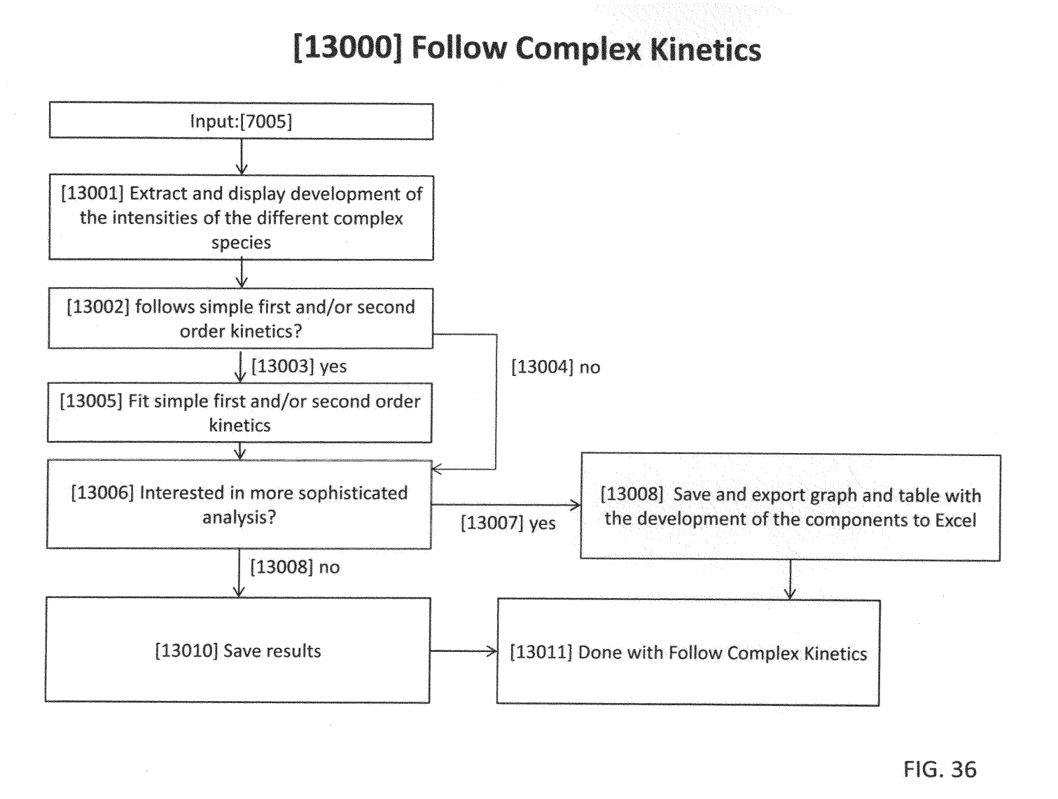

FIG. 36 shows a flow chart showing the steps in a sub-program 13000 for following complex kinetics. In some mass spectrometer experiments a sample is analyzed over time or at different concentrations of one or more components to evaluate the change in species depending on time or concentration. The following complex kinetics sub-program allows the user to analyze a time-evolution of mass spectra to determine changes in complexes and associated kinetics.

If the user decides after 7005 to follow complex kinetics, the sub-program 13000 extracts and displays the development of the intensities of each complex species 13001, for a series of mass spectra, which have been simulated as previously described. The user then determines whether or not the data follow simple first and/or second order kinetics 13001. If the user chooses 13003 to fit simple first and/or second order kinetics, the sub-program 13000 can be used to fit the simple first and/or second order kinetics 13005. After simple first and/or second order kinetics has been fit 13005 or the user is not interested 13004 in fitting simple first and/or second order kinetics, the user can then determine whether or not a more sophisticated analysis is desired. If the user is not interested 13008 in a more sophisticated analysis then the results are saved 13010 and the user is done 13011 with following complex kinetics. If the user is interested in a more sophisticated analysis 13007, then the graph and table containing the results of the development of the components are saved and exported 13008, for example, to Microsoft® Excel®. Then the user is done 13011 with following complex kinetics.

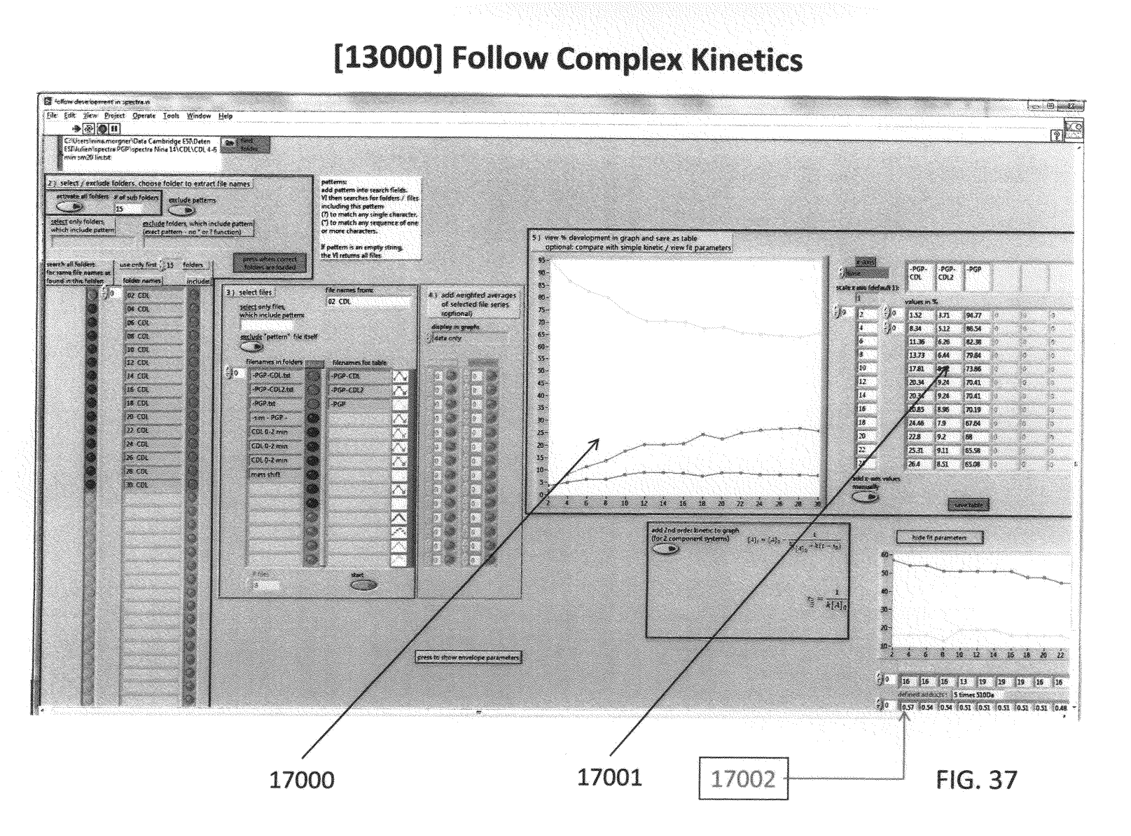

FIG. 37 shows a screen capture showing a user interface for the sub-program for following complex kinetics. Development and intensities of different complex species are shown in a graph 17000 and their corresponding numerical values are shown in a table 17001. This data can be exported to Excel® if the user wants a more sophisticated analysis. The user as well has the option to display 17002 the attachment as well as defined adduct parameters (if used) determined for the fit for each spectrum.

FIG. 38 shows a flow chart showing steps for smoothing a step-function. The smoothing of the step function can be called from within the find background sub-program 3000. For example, the program can call the program 1000a, which is used here as a sub-program. The user can input a smoothing constant n 1002. In one embodiment the 3002 sub-program then 1003 replaces each data point (i) by the average of the data points' interval:

[i−(n−1)/2, i+(n−1)/2)]

The smoothed step function is saved, applied and then 3003 the user determines if the background is too low.

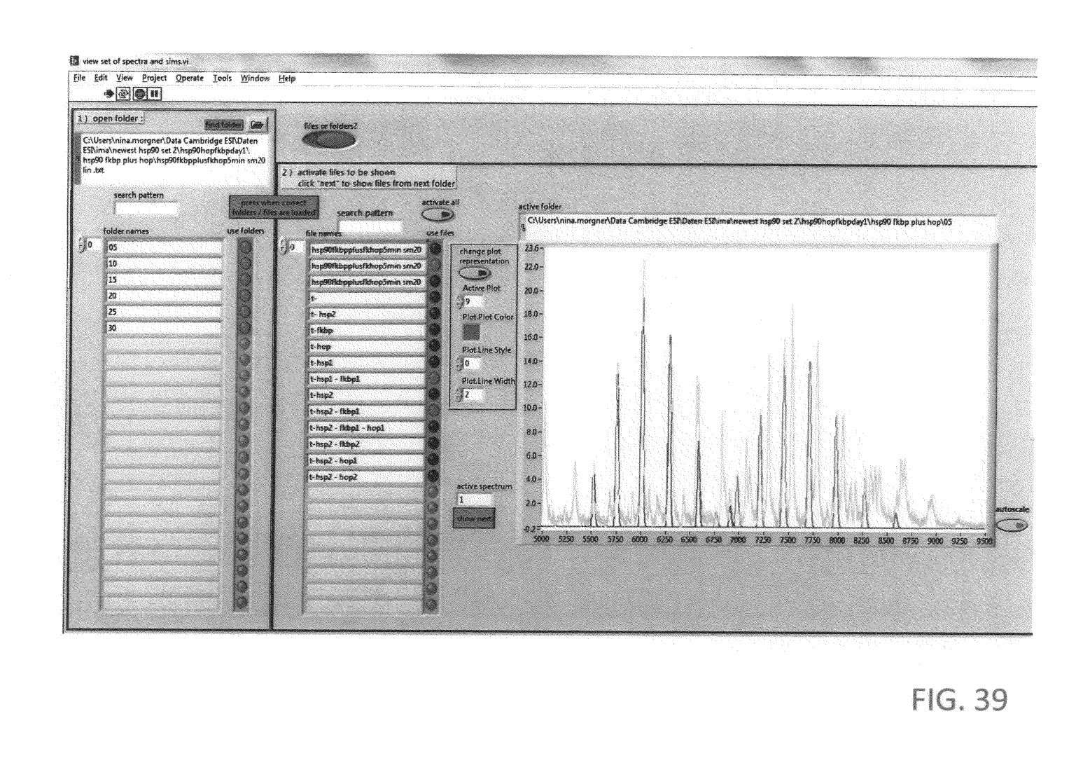

FIG. 39 depicts a screen shot of a sub-program that allows looking through a number of spectra (for example, one after the other) either all in one folder or in a set of folders. If looking through folders and several spectra are in each folder they can be shown simultaneously (for example the experimental spectrum and a simulation, shown simultaneously for comparison).

FIG. 40C depicts a list of sub-programs that may be included in an exemplary software package of the present disclosure we refer to as Massign and an order in which they may be used.

As shown with reference to FIGS. 1 through 34, the disclosed embodiments teach the steps of identifying peak series and determining mass-to-charge ratios; simulating charge state series; and assigning complexes, sub-complexes, and/or kinetics associated with the identified peak series. Unlike conventional software that is not suited for analysis of large complexes, the various programs and sub-programs disclosed herein allow for a user to analyze large complexes, along with their corresponding kinetics.

The processes described herein, and their component steps, may be implemented in hardware, software, firmware, or a combination thereof In the preferred embodiment(s), these processes are implemented in software or firmware that is stored in a memory and that is executed by a suitable instruction execution system. If implemented in hardware, as in an alternative embodiment, these processes can be implemented with any or a combination of the following technologies, which are all well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc. Thus, for example the processes described herein, and their component steps, may be implemented in a system comprising means for receiving an experimental mass spectrum, involving use of at least one computing device and at least one application executable in the at least one computing device, the at least one application implementing one or more of the embodiments described herein.

Any process descriptions or blocks in flow charts should be understood as representing modules, segments, or portions of code which include one or more executable instructions for implementing specific logical functions or steps in the process, and alternate implementations are included within the scope of the preferred embodiment of the present disclosure in which functions may be executed out of order from that shown or discussed, including substantially concurrently or in reverse order, depending on the functionality involved, as would be understood by those reasonably skilled in the art of the present disclosure.

The processes described herein may be implemented as a computer program, which comprises an ordered listing of executable instructions for implementing logical functions, can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can contain, store, communicate, propagate, or transport the program for use by or in connection with the instruction execution system, apparatus, or device. The computer-readable medium can be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM or Flash memory) (electronic), an optical fiber (optical), and a portable compact disc read-only memory (CDROM) (optical). Note that the computer-readable medium could even be paper or another suitable medium upon which the program is printed, as the program can be electronically captured via, for instance, optical scanning of the paper or other medium, then compiled, interpreted or otherwise processed in a suitable manner if necessary, and then stored in a computer memory.

### Assignment Example

We now provide an example of an assignment process of the present disclosure. This example is given for a spectrum of rotary ATPase from E. hirae, reported recently. “Mass Spectrometry of Intact V-Type ATPases Reveals Bound Lipids and the Effects of Nucleotide Binding,” by Zhou et al., Science 334(6054):380-385 (2011); See also, Appendix A hereto and incorporated herein. This ATPase has nine different subunits: A, B, C, D, E, F. G. I and K.

ATPases/synthases are large membrane complexes, consisting of two parts. The head is composed of three subunits A and B each, which alternate around the 6-membered ring. The second part includes a species dependent membrane embedded rotor ring, which transports protons. Prior to our investigation, the number of K subunits of the E. hirae rotor was ambiguous. It had been reported as 7 (EM) as well as 10 (X-ray crystallography). The peripheral stalk in this case consists of two subunits E and F. Due to the three-fold design of the head 1, 2, or maximally 3 stalks are present in ATPases. This ATPase was thought to have only one stalk, but this was not confirmed. So for our assignment, the number of stalks was varied between 1 and 3. Summing all possible combinations given the restriction in the head, peripheral stalks, and membrane ring, the intact complex could contain between 19 and 26 proteins.

Fitting of the peaks returned the component mass spectra (see FIG. 41A), the masses, and charge distributions of the subunits and sub-complexes in the spectrum, listed in SI Table S-1, Appendix A. Plotting masses obtained versus the charge states shows separation into two groups (inset FIG. 41B)—those species that group at lower charges are CID products, while the others are sub-complexes which form in solution. The solution complexes will form by dissociation or loss of subunits/sub-complexes, while CID complexes in almost all cases will form from one of the solution complexes via loss of a single subunit which is sometimes followed by the loss of a second subunit. In the next step we determined CID relationships as well as relationships between solution complexes. For the E. hirae ATPase a set of relations were identified, (listed in SI Tables S-2 and S-3, Appendix A).

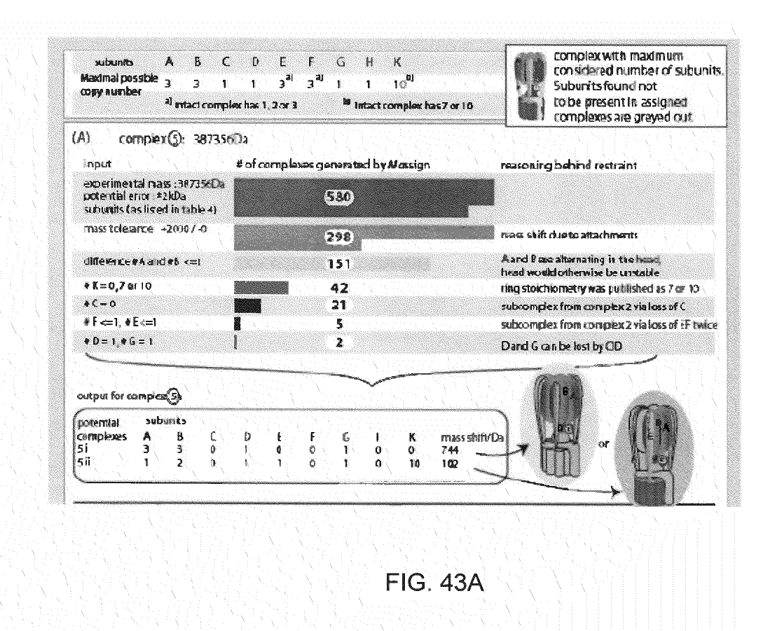

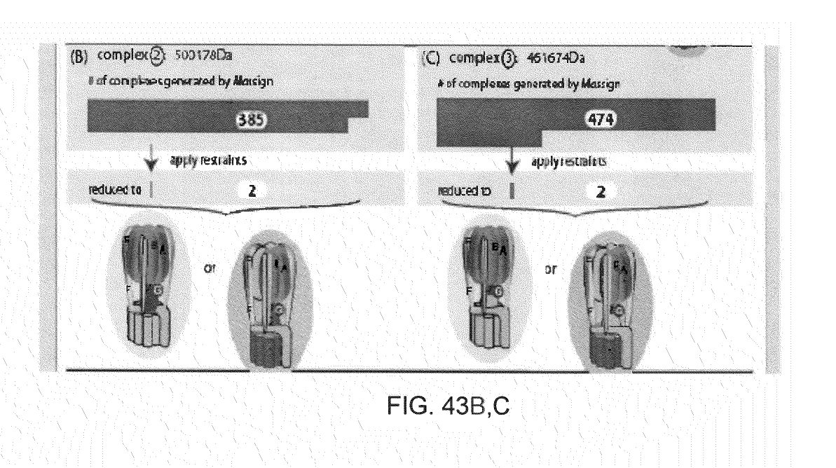

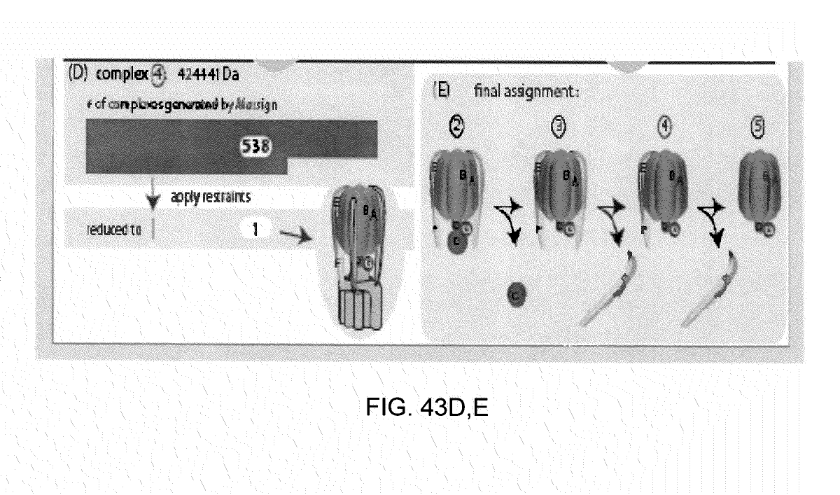

These relationships can be transferred into a connection network, of solution and CID complexes, as shown in FIG. 41B. While the complex can lose E or F (stalk proteins) in CID, the solution complexes show that a stalk is lost only as a pairwise interaction (E and F together). Three complexes show successively a mass difference consistent with the stalk (masses of 3-4 and 4-5), which confirms the existence of at least two stalks. These findings can be used as input to assign the sub-complexes to the masses. It is not possible to show the complete assignment process for all E. hirae ATPase sub-complexes. However we illustrate the process for four sub-complexes (FIG. 43), which we have assigned as solution complexes based on their charge/mass ratios (complexes 5, 4, 3, and 2; inset in FIG. 41B).

More particularly, FIG. 41 A depicts components of the mass spectra that were simulated. Masses and charge states were determined. Some complexes, however, are not yet identified and are therefore numbered 1-18. FIG. 41 B depicts a schematic used to derive values for Table S-1, Appendix A. The complexes separate by their charge/mass ratios into solution phase (green) and CID (orange) complexes. See inset, FIG. 41B. A potential connection network between the complexes observed was then constructed. All possible sub-unit combinations which could account for the mass difference between two complexes were then calculated with a mass tolerance set to ±1000 Da. The deviation between the theoretical sub-unit mass and the observed mass difference is shown in every case (black/gray). Those sub-unit combinations which are possible theoretically, but would not allow for a self-consistent set of complexes within the established rules of stoichiometry or connectivity (as listed in Table S-3, Appendix A) are greyed out, leaving the possible ones (black). In the particular example, the only candidate for the mass difference of complexes 5 and 6 is sub-unit G. The calculated mass difference is 213 Da greater than the theoretical protein mass. Possible candidates for mass difference between complexes 6 and 7 are ΔD or ΔFG. If complex 6 is derived from complex 5 via loss of sub-unit G, loss of ΔFG would break the stoichiometry rule since the complex cannot lose more than one G. Loss of D is the only remaining option. ΔFG is therefore shown in gray.

Our experience shows that for complexes in the mass range of several hundred kilodalton, one can expect mass shifts (difference between naked protein mass and measured complex mass (peak center)) up to 2 kDa. As a consequence, starting values allow for a deviation of 2 kDa between the experimental mass and the theoretical mass. During the assignment process, the early on assigned complexes defined the mass shift to be taken into account for later assignments will to be much smaller, simplifying the assignment (FIG. 42).

For every subunit, the maximum possible copy number is added as input into the software. In FIG. 42, we show the selection process of the mathematically possible subunit combinations, generated by the software to match the observed masses, based on the mass of the complex and the subunits. In particular, FIG. 42 shows the surface are per mass, calculated for globular proteins in the inset. The dotted lines indicate the area of interest for the assignment in the main panel of the figure. The mass shift of a complex stems from adducts attached to the surface, which correlates the mass shift with the mass in the main graph. For the assignment of E. hirae ATPase complexes, the default mass shift is 0-2000 Da (blue area). The first four complexes assigned in FIG. 43 (red crosses) allow the user to eliminate the range for the mass shifts to be expected for all complexes in this mass spectrum. The optimized mass shift range is shown in green and is used to eliminate potential assignments for the remaining complexes, which lie outside this range. Potential assignments, inside this range will be very few, usually only one. This complex can then be considered to have the correct assignment (blue crosses). The error bars shown are the errors determined for the masses, since the precision of the complex mass measurement will affect the range for the mass shift that has to be expected.

For complex 5 with a mass determined as 387 356 Da, the present method finds 580 possible subunit combinations given the default tolerance. Subsequently the number of possible complexes is reduced by adding connectivity and stoichiometry restraints into the software. These restraints for complex 5 reduce the number of potential complexes to two (FIG. 43A). The same strategy is applied to the other solution complexes. For complexes 2 and 3, the software output for both complexes is two possibilities (FIGS. 43B and 43C). The potential complexes are depicted in FIG. 43. A self-consistent set of complexes derived from each other can be seen for both sets of complexes. At this point it is not clear which solution is the correct one. Assignment of complex 4 then gives only one solution, which fits into only one set of solutions (FIG. 43D). This allows the unambiguous assignment of all four complexes as self-consistent set of solution complexes (FIG. 43E).

For the analysis of further complexes an additional restraint can be applied: The mass shift due to attachment of adducts can be of the order of 1 or 2 kDa, but for complexes in the same spectrum, the amount of adducts will be correlated. Therefore the default setting for the mass tolerance which to be allowed in the complex assignment process is 2 kDa at reduce the mass tolerance for the assignment of the remaining complexes. These first complexes to be assigned will often be the smallest or biggest ones, as mentioned earlier, but since in our example we already assigned four complexes (FIG. 43) we illustrate the effect using the complexes already assigned (FIG. 42). Complexes 2-5 now define the range of the expected mass shift for all E. hirae ATPase complexes in this spectrum. The mass shift allowed for the assignment can now be minimized accordingly.

If the assignment process does not produce a consistent set of complexes it is advisable for the user to retrace his/her steps and to reconsider if the restraints that were chosen for stoichiometry and connectivity could be wrong. It is worth noting, that the aim of this present disclosure is not to act as a black box, into which one inputs a spectrum and which then outputs assignments. Instead it can support the user in dealing with more and more complex sets of data, while allowing the user to stay in complete control of the entire process.

Attachment of Small Molecules

As mentioned earlier the quality and resolution of mass spectra can vary noticeably between spectra but in general the resolution is the same over the whole mass range for a single spectrum. Nevertheless we sometimes encounter mass spectra in which one or two peak series are much broader than the others. From experience we have found that it is worth paying attention to these irregularities. Peak series which appear to be noticeably broader than all other peak series present in the same mass spectrum can be expected to represent not one single sub-complex but a heterogeneous distribution of complexes very close in mass. While this can be due to truncations or PTMs (depending on the size of the distribution), the cases could in general be explained by a complex with varying amounts of ligands bound, which show a specific binding with certain sub-complexes. For ATPases we commonly observed binding of nucleotides to complexes containing the soluble head as well as ligands and/or nucleotides binding to complexes containing the membrane ring. In some cases the attachments leading to the broad peak features might be visible by means of shoulders in the peaks. In any case these features may be of importance if one wants to assign a complex to the observed mass and should therefore be kept in mind. While the general mass shift found for all complexes may be incorporated into the assignment strategy (as explained in the previous paragraph), these “complex specific” shifts can be factored in as mandatory “sub-units” of the complex. This may be important for example in the binding of six lipids and nucleotides to the membrane embedded C-ring, of Thermus thermophilus ATPase. “Mass Spectrometry of Intact V-Type ATPases Reveals Bound Lipids and the Effects of Nucleotide Binding,” by Zhou et al., Science 334(6054):380-385 (2011). This lipid and nucleotide binding was found to induce a mass shift of more than 4 kDa. This binding assignment was later confirmed by identification and quantitative analysis of the specifically bound lipids. If this had gone unnoticed, the assignment of the membrane containing complexes would have been impossible.

Quantitative Assignments

The simulation of the spectra of the present disclosure allows additionally the comparison of signal intensities represented in the component spectra to obtain quantitative information on the complex distribution. It is possible therefore to determine for example (de)stabilization effects due to changes in the sample environment (change of pH, addition of nucleotides, etc.) by comparing the intensities of different sub-complexes, under the same instrumental conditions. As seen from the forgoing, the present disclosure provides an assignment strategy which allows the qualitative and quantitative analysis of the mass spectra of heterogeneous, dynamic complexes. The assignment strategy presented here makes systematic use of masses, charge states, stoichiometry, and connectivity information. Overall using this method makes it possible to establish connectivity networks, assembly/disassembly pathways, and kinetic analysis and to study the reaction to change in solution conditions. This can not only establish K_{D}s, stable complexes in solution, connectivity, and stoichiometry but also highlight possible regulatory and allosteric interactions.

Although exemplary embodiments have been shown and described, it will be clear to those of ordinary skill in the art that a number of changes, modifications, or alterations to the disclosure as described may be made. All such changes, modifications, and alterations should therefore be seen as within the scope of the disclosure.

What is claimed is:

1. A method for analyzing mass spectra, comprising:
receiving an experimental mass spectrum from a spectrometer;
identifying peak series in an experimental mass spectrum and simulating the experimental mass spectrum by simulating the charge state series of different components determined from identifying peak series; and
assigning complexes and sub-complexes associated with the identified peak series.

2. The method of claim 1, wherein the spectrometer is one or more of an electrospray ionization mass spectrometer (ESI/MS) or a liquid chromatography mass spectrometer (LC/MS).

3. The method of claim 1, wherein the step of identifying peak series in the experimental mass spectrum includes determining charge state series present in the spectrum.

4. The method of claim 3, including determining masses from peak tops in the spectrum.

5. The method of claim 4, including first selecting one peak and varying the charge state of the selected peak and comparing a theoretical charge state distribution with all of the peaks in the experimental spectrum.

6. The method of claim 1, wherein the step of simulating the charge state series includes fitting each peak identified to a defined peak shape with a Gaussian distribution onset and either Gaussian or Lorentzian trailing edge.

7. The method of claim 7, wherein the fitting of each peak determines a midpoint of each peak in the charge state series.

8. The method of claim 8, wherein overlapping charge states are considered simultaneously.

9. The method of claim 9, further including displaying the simulated spectra simultaneously overlaid with the experimental spectrum.

10. The method of claim 1, wherein the step of simulating the charge state series determines a list of masses/charge distributions found in the spectrum, component spectra in the spectrum and an overall simulation of the experimental mass spectrum.

11. The method of claim 1, further including the steps of: a) smoothing and linearizing spectra in the mass spectrum; b) optionally combining spectra from step (a); c) subtracting background from the spectra; d) finding a mass series of the spectra; e) simulating component spectra individually; and f) minimizing deviation of a sum of simulated spectra from the experimental mass spectrum.

12. The method of claim 11, further including overlaying simulated spectra with the experimental spectrum to determine whether any parts of the experimental spectrum which have not been accounted for by the simulation, and repeating the steps of claim 11 as needed until all component spectra of the experimental mass spectrum have been simulated.

13. The method of claim 1, further including replacing the trailing edge of one or more simulated component spectra by a broadened version of the simulated peak.

14. The method of claim 1, wherein the step of assigning complexes and sub-complexes associated with the identified peak series includes distinguishing between complexes formed in solution and those formed via collision induced dissociation (CID).

15. The method of claim 14, further including using a mass/charge relation of complexes to separate between complexes formed in solution and those formed via collision induced dissociation (CID).

16. The method of claim 15, further including establishing precursor and product relationships based on mass/charge differences of the complexes.

17. The method of claim 14, further including determining an increase of a measured mass of a complex as compared to the mass of the naked complex.

18. The method of claim 14, further including support to determine the correct subunit combinations for the found masses, including determining a complete list of mathematically possible complexes which fall within allowed mass range close to the determined mass and reducing the list according to rules from user input and established rules determined from establishing precursor and product relationships based on mass/charge differences of the complexes.

19. A system for analyzing mass spectra, comprising:
at least one application executable in a computing device, the at least one application comprising:
logic that identifies peak series in an experimental mass spectrum and simulates the experimental mass spectrum by simulating the charge state series of different components determined from identifying peak series; and
assigns complexes and sub-complexes associated with the identified peak series.
